(19) United States
(12) Patent Application Publication
(10) Pub. No.: US 2004/0157213 A1
(43) Pub. Date: Aug. 12, 2004

(54) Fungal reverse transcriptases with enhanced capabilities

(76) Inventor: John C. Kennell, Saint Louis, MO (US)

Correspondence Address: SAINT LOUIS UNIVERSITY, OFFICE OF RESEARCH ADMINISTRATION, 3556 CAROLINE STREET, ST. LOUIS, MO, 63104, US

(21) Appl. No.: 10/765,456

(22) Filed: Jan. 26, 2004

Related U.S. Application Data
(60) Provisional application No. 60/442,885, filed on Jan. 27, 2003.
(60) Provisional application No. 60/459,775, filed on Apr. 2, 2003.

(51) Int. Cl. C12Q 1/70; C07H 21/04; C12N 9/22

(52) U.S. Cl. 435/5; 435/69.3; 435/320.1; 435/199; 536/23.2; 435/325

(57) ABSTRACT

Disclosed are reverse transcriptase molecules which are capable of synthesizing nucleic acids using a template polynucleotide and a nucleotide primer, such that the nucleotide primer provides a free 3′-OH and the nucleotide comprising the free 3′-OH does not need to be paired with a complementary base on the template polynucleotide. Preferred reverse transcriptases are pFOXC2-RT and pFOXC3-RT, which are derived from the mitochondria of the fungus Fusarium oxysporum. Also disclosed are improved methods of making long or short cDNAs using the reverse transcriptases derived from the mitochondria of the fungus Fusarium oxysporum. Also disclosed are polynucleotides and recombinant vectors that encode the reverse transcriptases derived from the mitochondria of the fungus Fusarium oxysporum.

### PARENT CASE TEXT

This application claims benefit of priority to U.S. Provisional Patent Application No. 60/442,885, filed Jan. 27, 2003, and U.S. Provisional Patent Application No. 60/459,775, filed Apr. 2, 2003.

### GOVERNMENT SUPPORT CLAUSE

This work was supported by a grant from the U.S. National Science Foundation, grants number 9982775 and MCB-0196483. The U.S. Government has certain rights in this invention.

### SEQUENCE LISTING

A paper copy of the sequence listing and a computer readable form of the same sequence listing are appended below and herein incorporated by reference. The information recorded in computer readable form is identical to the written sequence listing, according to 37 C.F.R. 1.821 (f).

### BACKGROUND OF THE INVENTION

1. Field of the Invention

The present invention relates generally to reverse transcriptases and DNA polymerases useful in research and diagnostic applications.

2. Description of Related Art

All references cited in this specification are hereby incorporated by reference. The discussion of references is merely intended to summarize the assertions made by their authors and no admission is made that any reference constitutes prior art. Applicants reserve the right to challenge the accuracy and pertinence of the cited references.

Blackburn, E. H. (1999) Telomerases. In: The RNA World, second edition (ed. Gesteland, R. F., Cech, T. R., Atkins, J. F.) Cold Spring Harbor Laboratory Press, Coldspring Harbor, N.Y. pp. 609-635

Chen, B. and Lambowitz, A. M. (1997) De novo and DNA primer-mediated initiation of cDNA synthesis by the Mauriceville retroplasmid reverse transcriptase involve recognition of a 3′ CCA Sequence. J. Mol. Biol. 271, 311-332.

Curcio, M. J. and Garfinkel, D. J. (1991) Single-step selection for Ty1 element retrotransposition. Proc. Natl. Acad. Sci. 88, 936-940.

Dombroski, D. A., Feng, Q., Mathias, S. L., Sassaman, D. L., Scott, A. F., Kazazian, H. H., Jr., and Boeke, J. D. (1994) An in vivo assay for the reverse transcriptase of human retrotransposon L1 in Saccharomyces cerevisiae. Mol. Cell. Bio. 14, 4485-4492.

Eickbush, T. H. (1994) Origin and evolutionary relationships of retroelements. In: Evolutionary Biology of Viruses (ed. Morse, S. S.), Raven Press, New York pp. 121-157.

Eickbush, T. H. (1997) Telomerase and Retrotransposons: Which Came First? Science 277, 911-912.

Eickbush, T. H. (2002). R2 and related site-specific non-long terminal repeat retrotransposons. In: Mobile DNA II (ed. Craig N. L, Craigie R, Gellert M, and Lambowitz A. M.), Raven Press, Washington D.C. pp. 813-835.

Eickbush, T. H., Malik H. S. (2002) Origins and evolution of retrotransposons. In: Mobile DNA II (ed. Craig N. L, Craigie R, Gellert M, and Lambowitz A. M.), Raven Press, Washington D.C. pp. 1111-1144.

Hansen, J. L., Long A. M. and Schultz, S. C. (1997) Structure of the RNA-dependent RNA polymerase of poliovirus. Structure. 5, 1109-1122.

Kajikawa, M. and Okada, N. (2002) LINEs mobilize SINEs in the eel through a shared 3′ sequence. Cell 111, 433-444.

Kennell, J. C. (2000) Educational benefits associated with Service-learning projects in biology curricula. In: American Association of Higher education: series on Service-Learning-Biology. (ed. Brubaker, D. C., Ostroff, J. H. and Zlotkowski, E.) American Associaion of Higher Education. Washington, D.C. pp 7-23.

Kistler, H. C. and Leong, S. A. (1986) Linear plasmidlike DNA in the plant pathogenic fungus Fusarium oxysporum f. sp. conglutinans. J. Bacteriol. 167, 587-593.

Kistler, H. C., Benny, U. and Powell, W. A. (1997) Linear mitochondrial plasmids of Fusarium oxysporum contain genes with sequence similarity to genes encoding a reverse transcriptase from Neurospora spp. Appl. Environm. Microbiol. 63, 3311-3313.

Kuiper, M. T. R., Sabourin, J. R. and Lambowitz, A. M. (1990) Identification of the reverse transcriptase encoded by the Mauriceville and Varkud mitochondrial plasmids of Neurospora. J. Biol. Chem. 265, 6936-6943.

Loeb, D. D. and Ganem, D. (1993) Reverse transcriptase pathway of the hepatitis B viruses. In: Reverse Transcriptase (ed. Salka, A. M. and Goff, S. P.), Cold Spring Harbor Laboratory Press. Cold Spring Harbor. pp. 329-355.

McEachern, M. J., Krauskopf, A. and Blackburn E. H. (2000) Telomeres and their control. Annu. Rev. Genet. 34, 331-358.

Moran, J. V., Holmes, S. E., Naas, T. P., DeBerardinis, R. J., Boeke, J. D. and Kazazian, H. H. (1996) High frequency retrotransposition in cultured mammalian cells. Cell 87, 917-927.

Nakamura, T. M. and Cech, T. R. (1998) Reversing time: origin of telomerase. Cell 92, 587-590.

Nissley, D. V., Garfinkel, D. J. and Strathern, J. N. (1996) HIV reverse transcription in yeast. Nature 380, 30-32.

Okada, N. Hamada, M. Ogiwara, I. and Ohshima, K. (1997) SINEs and LINEs share common 3′ sequences: a review. Gene 205, 229-243.

Pardue, M. L. and DeBaryshe. (2002). Telomeres and transposable elements. In: Mobile DNA II (ed. Craig N. L, Craigie R, Gellert M, and Lambowitz A. M.), Raven Press, Washington D.C. pp. 870-887.

Qadri, I. and Siddiqui A. (1999) Expression of Hepatitis B virus polymerase in Ty1-his3AI retroelement of Saccharomyces cerevisiae. J. Biol.Chem. 274, 31359-31365.

Salas, M. (1991) Protein-priming of DNA replication. Annu. Rev. Biochem. 60, 39-71.

Walther, T. Ch. and Kennell, J. C. (1999) Linear mitochondrial plasmids of Fusarium oxysporum are novel, telomere-like retroelements. Molecular Cell 4:229-238.

Wang, H., Gilley, D. and Blackburn E. H. (1998) A novel specificity for the primer-template pairing requirement in Tetrahymena telomerase. EMBO 17, 1152-1160.

Wang, H. and Lambowitz, A. M. (1993) The Mauriceville plasmid reverse transcriptase can initiate cDNA synthesis de novo and may be related to the progenitor of reverse transcriptases and DNA polymerases. Cell 75,1071 -1081.

Wang, H., Kennell, J. C., Kuiper, M. T. R., Sabourin, J. R., Saldanha, R. and Lambowitz, A. M. (1992) The Mauriceville plasmid of Neurospora crassa: characterization of a novel reverse transcriptase that begins cDNA synthesis at the 3′ end of template RNA. Mol. Cell Biol. 12,5131-5144.

Wang, G. H. and Seeger, C. (1992) The reverse transcriptase of hepatitis B virus acts as a protein primer for viral DNA synthesis. Cell 71, 663-670.

Zoulim, F. and Seeger, C. (1994) Reverse transcription in hepatitis B viruses is primed by a tyrosine residue of the polymerase. J. Virol. 68, 6-13.

### Reverse Transcriptase

A method commonly used in molecular biology is the conversion of RNA to complementary DNA (“cDNA”). This procedure, called reverse transcription, may be used in multiple applications, including generating a cDNA library and profiling gene expression patterns, as in reverse transcription-polymerase chain reaction (“RT-PCR”). The ability to create cDNAs and make cDNA libraries have made possible the study and discovery of many biologically important molecules and processes.

The driving force in the construction of cDNA sequences is the reverse transcriptase enzyme (“RT”). Commonly found in retroviruses, a group of viruses whose genetic material consists of a single-stranded RNA, reverse transcriptase enzymes synthesize cDNA sequences, which are then capable of integrating into the host cell genome. Complementary DNA technology utilizes in vitro the action of reverse transcriptase present in retroviruses. The most commonly used method by which a mRNA template is copied into a cDNA uses an oligo-dT primer or randomly synthesized nucleotide primer, which anneals to the RNA template using the rules of base-complementarity. The —OH group of the 3′ terminal nucleotide of the bound oligonucleotide serves as the initiation point for polynucleotide strand synthesis. The proper base-pairing of this 3′-nucleotide is required by most of the known reverse transcriptases to initiate cDNA synthesis. Once initiated, reverse transcriptase catalyzes the addition of deoxynucleotides to the growing cDNA strand. The template RNA strand is then removed from the newly created cDNA strand by the action of RNase H. The newly created cDNA strand is generally referred to as the “minus-strand” or “first strand”. Alternatively but not exclusively, the RNA template strand may be displaced by a DNA polymerase, which may be added to the reaction mixture to produce the plus-strand. The plus-strand is then formed through the action of either the RT or another DNA polymerase to produce a double stranded cDNA molecule. This step is called the topping reaction. The resultant double-stranded DNA molecules can then be ligated into a plasmid vector, packaged into a virus for cloning, or likewise manipulated for further use.

Some problems associated with current RT technology relate to the cloning or amplification of RNA sequences using custom made RT primers that contain specific sequences. As mentioned above, current RT technology requires that the 3′-OH containing nucleotide of the primer (i.e., the 3′ nucleotide) be stably hydrogen bound to the template DNA or RNA in order for strand synthesis to occur. Given the presence of single nucleotide polymorphisms (“SNPs”) in most genomes as well as published errors in sequences, the reliance on published sequences to make custom RT primers runs the risk of having RT primers that are unable to generate a cDNA due to a 3′-nucleotide mismatch, and therefore yield a false negative.

Additionally, there is a growing interest in the regulatory role of small RNAs in eukaryotic systems. These RNAs, alternatively called micro RNAs (“miRNAs”), small interfering RNAs (“siRNAs”), or small temporal RNAs (“stRNAs”), are very small (approximately 21 to 25 nt) and are not poly-adenlyated. There is growing evidence of the importance of these small RNAs in the regulation of gene expression and cellular differentiation via RNA interference (“RNAi”). Due to their small size and lack of a reliable primer binding site, the cloning these small RNAs by conventional methods is problematic. The use of a RT, which efficiently uses self-primed RNA templates, e.g., via a snapback mechanism, or one that has a loose or low primer binding specificity, would greatly facilitate the copying and cloning of these RNAs.

### Mitochondrial Retroelements

Retroelements are genetic elements that replicate via reverse transcription. They are highly successful molecular parasites and appear to be ubiquitous among eukaryotic organisms, comprising up to 70% of genomic DNA in some species. As a group, retroelements represent a diverse collection of genetic elements that employ a wide variety of replication strategies. Th ese include retroviruses that move from cell to cell (i.e. Human Immunodeficiency Virus) or within genomes [i.e. Long Terminal Repeat (LTR) elements like Ty1 of yeast and non-LTR elements such as the human L1], as well as autonomously replicating retroplasmids (i.e. Mauriceville plasmid of Neurospora). The success of these elements is phenomenal considering the coding capacity of most retroelements is relatively small, usually less than a dozen gene products. Although they depend on their host for many functions, retroelements often have a broad host range.

The pFOXC plasmids, pFOXC2, and pFOXC3, whose encoded RTs have novel and surprising activities, which are the subject of the instant invention, were initially discovered in mitochondria of two different strains of the fungal plant pathogen Fusarium oxysporum (Kistler and Leong, 1986). The plasmids are linear double-stranded DNAs of approximately 1.9 kb and contain a single long open reading frame (“ORF”) that encodes a reverse transcriptase (“RT”; see Kistler et al., 1997; Walther and Kennell, 1999). These retroplasmids have a unique “clothespin” structure, which includes a hairpin at one terminus and a telomere-like repeat of a 5 bp sequence (ATCTA) at the downstream terminus. The number of repeats is not constant, suggesting that the 3′ end of the plasmid DNA is in flux and that specific mechanisms may be involved in the generation and maintenance of the repeats. A hypothetical model for the replication of pFOXC plasmids, which is based on the structural organization of the plasmid DNA, analysis of reverse transcriptase assays using mt RNP particles and the characterization of the plasmid transcript, is described in Walther and Kennell, 1999, which is herein incorporated by reference.

The discovery of an active reverse transcriptase encoded by genetic elements having telomere-like repeats suggests that the pFOXC plasmids are related to elements that were the evolutionary precursors of the ribonucleoprotein complex known as telomerase. At its core, telomerase is composed of a reverse transcriptase (TERT) and a telomerase RNA (TER) that is used as a template for the synthesis of short, often G-rich, repeats at the 3′ end of eukaryotic chromosomes (Blackburn, 1999; McEachern et al., 2000). In addition, the 3′ repeats of the pFOXC plasmids bear a striking resemblance to 3′ tails of certain long and short interspersed elements (LINEs and SINEs; Okada et al., 1997). For example, in the eel genome, LINE and SINE elements share the same 5 bp repeat (TGTAA) at their 3′ end (Kajikawa and Okada, 2002).

The mechanism that RTs use to initiate cDNA synthesis is also an important characteristic as it often relates to the mode of replication of the element. For example, non-LTR retrotransposons use the 3′ OH of a nicked DNA target site to initiate cDNA synthesis, a mechanism called target-primed reverse transcription (TPRT; Eickbush, 2002). Interestingly, the Mauriceville-RT can initiate cDNA synthesis de novo (without a primer; Wang and Lambowitz, 1993) which suggests that it may be mechanistically related to RNA-dependent RNA polymerases—nucleotide polymerases with the greatest sequence and structural similarity to reverse transcriptases (Hansen et al., 1997).

### SUMMARY OF THE INVENTION

The present invention is based upon the surprising discovery that pFOXC2 and pFOXC3 reverse transcriptases, which are derived from a mitochondrial retroplasmid of the fungus Fusarium oxysporum, are able to catalyze nucleic acid polymerization or synthesize polynucleotides using primers that contain mismatched nucleotides at the 3′ end of the primer. Nucleic acid polymerization may involve either the synthesis of DNA using an RNA template or a DNA template. The primer may be a distinct oligonucleotide of at least 2 nucleotides in length, or it may be a portion of the template that has snapped back upon itself in a hair pin-like structure. The primer may by RNA or DNA, or a combination thereof.

The inventor has succeeded in developing an in vitro reverse transcriptase system to study the mechanism of cDNA synthesis catalyzed by pFOXC2 and pFOXC3 reverse transcriptases (hereinafter referred to as “pFOXC-RT”). It was discovered that the mechanism of polynucleotide polymerization and the characteristics of the templates and products of the polymerization catalyzed by the pFOXC-RT distinguish the pFOXC-RT from previously characterized viral RTs (e.g., MMLV-RT and AMV-RT) and previously characterized fungal mitochondrial RTs (e.g., Mauriceville and Varkud). For example, it is herein disclosed that cDNA synthesis using pFOXC-RT can be initiated using the 3′ hydroxyl of a RNA which can snapback upon itself, a feature not available to known viral RTs. Furthermore, the cDNA products generated by the pFOXC-RT on primer-template combinations appear to be larger than equivalent cDNAs synthesized by conventional RTs, MMLV-RT and AMV-RT.

Also, it is herein disclosed that pFOXC-RT is capable of utilizing a DNA primer. Although this is common for most enzymes that polymerize DNA (i.e. DNA-dependent DNA polymerases and reverse transcriptases), previous studies have shown that the closely-related reverse transcriptase encoded by the Mauriceville and Varkud mitochondrial plasmids of Neurospora do not readily use a DNA primer. Additional novel characteristics of pFOXC-RT discovered by the inventor include: (1) pFOXC-RT readily uses the 3′ OH of RNA templates to prime cDNA synthesis, whereas the Mauriceville-RT rarely uses RNA primers and appears to depend on a specific RNA sequence, rather than base-pairing of the 3′ end of the RNA primer (Wang and Lambowitz, 1993); (2) pFOXC-RT is able to use DNA primers that anneal at an internal region of the transcript, whereas the Mauriceville-RT cannot (Wang et al., 1992; Chen and Lambowitz, 1997); (3) pFOXC-RT is able to copy DNA templates, while the Mauriceville-RT cannot; (4) treatment of pFOXC-containing mt RNPs with micrococcal nuclease results in RT preparations free of endogenous RNAs or DNAs (at least, they are not used as primers for cDNA synthesis), whereas micrococcal nuclease-treated Mauriceville-RT preparations contain endogenous cDNA products that are used as primers for reverse transcription (Wang et al., 1992); (5) and pFOXC-RT has low selectivity for specific RNAs, whereas the Mauriceville RT highly prefers RNAs having a 3′ terminal CCA sequence (Chen and Lambowitz, 1997).

Having discovered that pFOXC-RT makes more efficient use of DNA primers that anneal to the 3′ terminus of RNAs, the inventor envisions that pFOXC-RT may be useful for detecting or quantifying highly variable RNAs, such as Retro or RNA viruses, which potentially could have mismatches with DNA primers used in cDNA synthesis. Having discovered that pFOXC-RT appears to be adept at using snapbacked RNAs, the inventor envisons that pFOXC-RT may be (i) useful in the cloning of hairpin molecules, such as siRNA; (ii) more proficient at carrying out second strand synthesis, either by eliminating steps in the generation of double-stranded DNAs for cloning, or potentially being more proficient at copying the 5′ end of RNAs; and (iii) useful in the analysis of non-polyadenylated RNAs in microarray experiments (e.g., prokaryotic RNAs for bacteriological assays, histone RNAs, snRNAs, and the like).

Thus, the invention is drawn to an isolated and purified pFOXC-RT polypeptide which has reverse transcriptase (RT) and DNA polymerization activity and is capable of synthesizing a polynucleotide in the presence of a free 3′-OH of a nucleotide that may or may not be paired with a complementary nucleotide of a template strand. A preferred pFOXC-RT polypeptide comprises a sequence that is at least 88% identical to a pFOXC2 or pFOXC3 sequence, as exemplified in SEQ ID NO:1 (pFOXC2) or SEQ ID NO:2 (pFOXC3), respectively.

In another embodiment, the invention is drawn to polynucleotides that encode a pFOXC-RT polypeptide, which has reverse transcriptase (RT) and DNA polymerization activity and is capable of synthesizing a polynucleotide in the presence of a free 3′-OH of a nucleotide that may or may not be paired according to the art recognized base-pairing rules with a complementary nucleotide of a template strand. (The art recognized base-pairing rules stipulate that adenine forms hydrogen bonds with the complementary base thymine or uracil and guanine forms hydrogen bonds with cytosine.) It is also recognized in the art that various organisms and organelles utilize different genetic codes, therefore the instant polynucleotides may utilize a universal genetic code or a mitochondrial genetic code. A preferred polynucleotide comprises a sequence as set forth in any one of SEQ ID NO:3-6. SEQ ID NO:2 and 3 represent polynucleotides that encode SEQ ID NO:1 and 2, respectively, utilizing the Universal genetic code, in which U/TGG codes for tryptophan and U/TGA is a stop codon. SEQ ID NO:4 and 5 represent polynucleotides that encode SEQ ID NO:1 and 2, respectively, utilizing the Mitochondrial genetic code, in which U/TGA and U/TGG both code for tryptophan. In another embodiment, the invention is drawn to plasmids, which may be either circular or linear, and other vectors that comprise any polynucleotide that encodes a pFOXC-RT. The polynucleotide may be operably linked to a promoter.

In another embodiment, the invention is drawn to cells and other in vitro or in vivo systems, which comprise the instant polynucleotide. The cells and systems are useful in the production of the instant pFOXC-RT polypeptide. Preferred systems or cells include bacteria, such as E. coli, yeast, such as Pichia spp. and Saccharomyces spp., the bacculovirus expression system, which utilizes insect cells, mammalian cell expression systems, and transgenic plant and animal systems.

In yet another embodiment, the invention is drawn to in vitro methods of making a polynucleotide (e.g., CDNA) comprising the steps of mixing a template polynucleotide with the instant pFOXC-RT in the presence of deoxynucleotides and magnesium. The template may be any RNA or DNA. A separate oligonucleotide primer may or may not be present, since the instant pFOXC-RTs may prime strand synthesis using a snapping-back mechanism, in which the 3′ OH is provided by the template strand snapping back upon itself. Preferred reaction conditions are pH 8.2, 42° C., 10-20 mM MgCl₂, and no salt.

In yet another embodiment, the invention is drawn to a method of isolating pFOXC-RT protein from a fungal mitochondrion, wherein the pFOXC-RT is of sufficient purity to be used in the in vitro synthesis of DNA.

The invention is also drawn to methods of making a pFOXC-RT using a heterologous (i.e., non-Fusarium oxysporum) protein expression system. Many heterologous protein expression systems are known in the art, including the Pichia pastoris system, E. coli and other bacterial systems, bacculovirus-insect cell systems, mammalian cells, and the like. Preferred heterologous systems are the yeast Saccharomyces and E. coli.

In yet another embodiment, the invention is drawn to antibodies that react specifically to epitopes of the instant pFOXC-RT. The antibodies are envisioned to be useful in the preparation, purification or isolation of the instant pFOXC-RT, and in the disruption of pFOXC-RT activity.

### BRIEF DESCRIPTION OF THE DRAWINGS

FIG. 1 depicts a schematic of a polynucleotide “minus-strand” synthesis reaction in which the 3′ nucleotide of the oligonucleotide primer is mismatched (i.e., does not base-pair with the template strand).

FIG. 2 depicts an alignment of the pFOXC2-RT (SEQ ID NO:1) and the pFOXC3-RT (SEQ ID NO:2) using the Clustal W program.

FIG. 3: In vitro reverse transcription assays using the pFOXC-RT and MMLV-RT. Reactions using mt RNP particles isolated from pFOXC3-containing strains digested with micrococcal nuclease (MN; lanes 1-5) or MMLV-RT (lanes 7-10). Lane 1, no exogenous RNA. Lanes 2-5 and 7-10, reactions containing a 93 nt in vitro synthesized RNA that corresponds to the 3′ terminus of the pFOXC3 plasmid transcript (C3:2r RNA). Reactions were carried out with (lanes 4, 5, 8 , 10) or without (lanes 2, 3, 9, 10) a 34 nt oligonucleotide that is complementary to 25 nt at the 3′ end of the in vitro RNA. Following cDNA synthesis, products were incubated with RNase A (lanes 3, 5, 8, 10) or left untreated (lanes 1, 2, 5, 7, 9), prior to electrophoresis in a 6% polyacrylamide gel containing 8M urea. Numbers on the left indicate the size (nts) of 100-bp and Sau3AI fragments of pBS(−) (M, lane 6) molecular weight markers. Numbers on the right indicate the size of the ³²P-labeled cDNA products as well as a schematic drawing of the products (red=C3:2r RNA, blue=cDNA, black=oligonucleotide).

FIG. 4: Extension of a 37 nt oligonucleotide (3R) by the MN-pFOXC-RT in reactions lacking an RNA template. Panel A: Unlabeled 3R oligo was used in in vitro cDNA reactions with MN-pFOXC-RT containing 0.33 μM ³²P-dATP and either 20 μM dCTP, dGTP and TTP (lane 1), no additional nucleotides (lane 2), 100 μM dideoxyTTP (lane 3), or 20 μM dCTP, TTP and 100 μM dideoxyGTP (lane 4). Lane 5 (farthest to the right) contains oligonucleotide 3R, labeled with ³²P-γ-ATP using polynucleotide kinase. Sizes are indicated on the left, which are based on DNA size standards (not shown). Panel B: The sequence and length of predicted extension products for each reaction is indicated for the three most favorable base-pairings. Nucleotides added during polymerization are lowercase (and are only indicated for the bottom primers). Vertical lines indicate Watson:Crick base pairing and a colon indicates potential G-T pairing.

FIG. 5: Determination of optimal conditions and specificity of the MN-treated pFOXC-RT using exogenous templates or template/primer substrates. A. MgC12 optimum using poly rC and oligo gG. B. Specificity of the MN-treated pFOXC3-RT. MN-treated pFOXC-RT was used in reactions containing total mitochondrial RNA isolated from pFOXC3-containing strain. Labeled products were used as a probe in a Southern hybridization to a blot containing EcoRI-digested mtDNA from the same strain. Arrow indicates hybridization to the 1.9 kb pFOXC3 DNA. C. Map of pFOXC2 and pFOXC3 showing the region used to produce in vitro RNAs C3:2R and C3:3R. E=EcoRI, E5=EcoRV, Bg=BglII, X=XmaI. D. Complementary DNA synthesis using C3:2R RNA as template in reactions having different KCl and MgCL₂ concentrations (in mM).

FIG. 6: Comparison of cDNA synthesis using AMV, MMLV and pFOXC-RT with different template:primer substrates. All reactions include the 98 nt C3:3R in vitro RNA that corresponds to the 3′ end of the pFOXC3 DNA having three copies of the five bp repeat (dashed line). Lanes 1, 4, and 7 lack a DNA primer; Lanes 2, 5 and 8 contain the oligonucleotide int (i; open box) that has 23 nt of complementarity to an internal region of the template; and lanes 3, 6 and 9 contain oligonucleotide 2c having 10 nt of complementarity to the 3′ end of the template. Drawings to the right of the image indicate the expected products.

FIG. 7: Comparison of cDNA synthesis with pFOXC-RT and MMLV-RT using primers having limited complementarity to 3′ end of the RNA template. All reactions contain 98 nt C3:3R RNA that was pre-annealed to either oligo 2R, 1R+AT (1Rat), 1R or INT (I) having 10, 7, 5 or 23 nt of complementarity to the template. Reactions were carried out under optimal buffer conditions containing ³²P-dATP at 25° C. for 20 minutes followed by a 10 minute chase. Size of pBS-Sau31 and pBS-AluI restriction fragments are shown on the right and estimated of cDNA products on the right. The base-pairing alignments predicted by the size of the products are shown below the figure and indicated with arrowheads. The grey arrowhead indicated a higher molecular weight band detected in the pFOXC-RT reaction containing 1R primer.

FIG. 8: In vitro reverse transcription assays using the pFOXC-RT and MMLV-RT. Reactions using mt RNP particles isolated from pFOXC3-containing strains digested with micrococcal nuclease (MN; lanes 1-5) or MMLV-RT (lanes 7-10). Lane 1, no exogenous RNA. Lanes 2-5 and 7-10, reactions containing a 93 nt in vitro synthesized RNA that corresponds to the 3′ terminus of the pFOXC3 plasmid transcript (C3:2r RNA). Reactions were carried out with (lanes 4, 5, 8, 10) or without (lanes 2, 3, 9, 10) a 34 nt oligonucleotide that is complementary to 25 nt at the 3′ end of the in vitro RNA. Following cDNA synthesis, products were incubated with RNase A (lanes 3, 5, 8, 10) or left untreated (lanes 1, 2, 5, 7, 9), prior to electrophoresis in a 6% polyacrylamide gel containing 8 M urea. Numbers on the left indicate the size (nts) of 100-bp and Sau3AI fragments of pBS(−) (M, lane 6) molecular weight markers. Numbers on the right indicate the size of the ³²P-labeled cDNA products as well as a schematic drawing of the products (dashed line=C3:2r RNA, box=oligonucleotide).

### DETAILED DESCRIPTION OF THE INVENTION

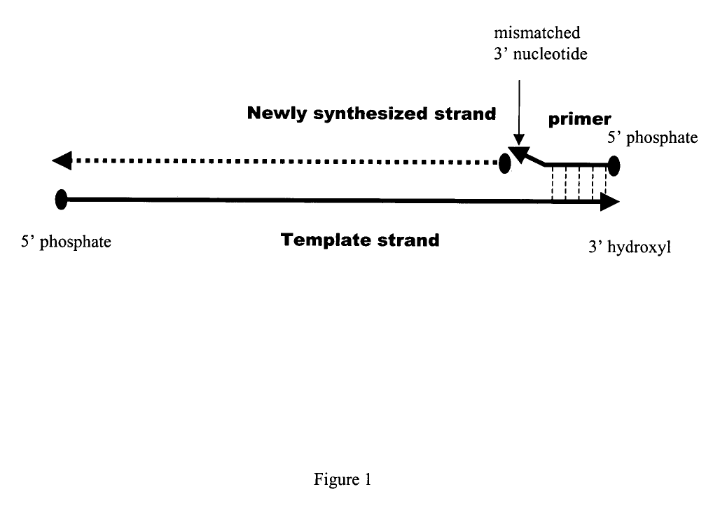

With the exception of the Neurospora sp. Mauriceville mitochondrial reverse transcriptase, which does not require a free nucleotide 3′-OH to prime DNA synthesis, reverse transcriptases, as well as other DNA polymerases, require a free nucleotide 3′-OH to prime DNA synthesis, wherein it is required that the 3′ nucleotide is hydrogen-bonded to a complementary base according to the art-recognized base-pairing rules (supra). The inventor of the instant invention has made the surprising discovery that Fusarium oxysporum mitochondrial reverse transcriptases (“pFOXC-RTs”) are capable of catalyzing the synthesis of polynucleotides utilizing a polynucleotide template (DNA or RNA) and a free 3′-hydroxyl group of a nucleotide base, which is not required to be hydrogen-bonded to a complementary base (i.e., a “non-base paired” or “mismatched” nucleotide; see FIG. 1). However, this does not preclude the situation in which the 3′ nucleotide of the primer transiently binds through dipole-dipole interaction with a non-complementary base on the template strand (i.e., A-C binding or G-T binding). This novel and surprising attribute enables the pFOXC-RT to be used in novel polynucleotide polymerization applications. However, while a mismatched 3′ nucleotide may be used during polynucleotide strand synthesis catalyzed by the instant pFOXC-RT, the polynucleotide primer (snapped back portion of the template or independent oligonucleotide) must be stably associated with the template strand by complementary base-pairing.

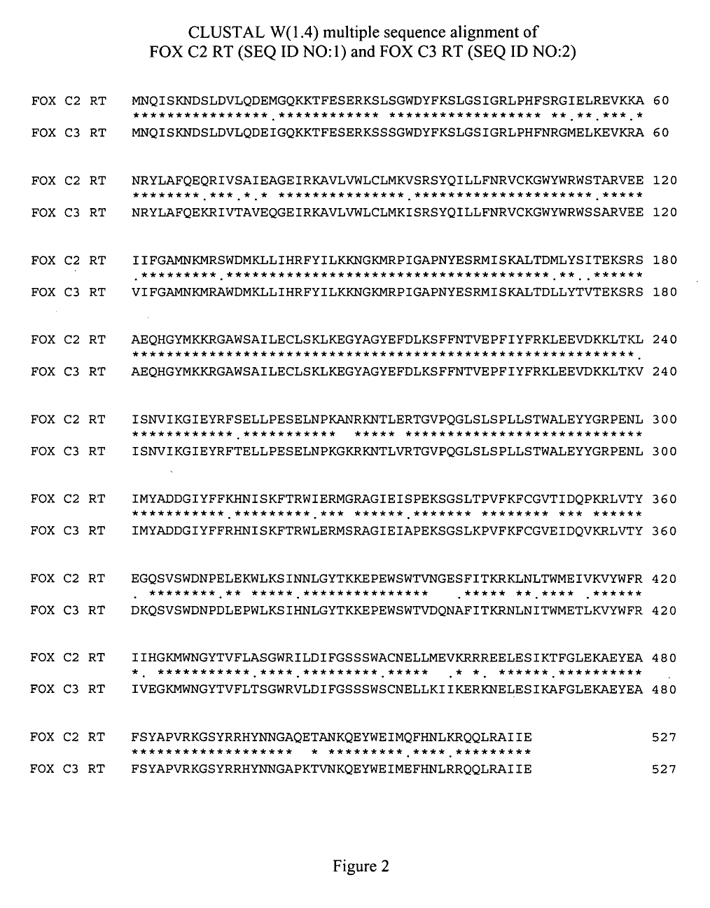

The naturally occurring Fusarium oxysporum mitochondrial reverse transcriptases are encoded by linear mitochondrial plasmids known as pFOXC2 and pFOXC3. Examplary polynucleotide sequences, which encode the pFOXC2 and pFOXC3 RTs having amino acid sequences set forth respectively in SEQ ID NO:1 and SEQ ID NO:2, and which utilize the Universal genetic code, are depicted in SEQ ID NO:3 and SEQ ID NO:4, respectively. The nucleotide sequences, which encode the pFOXC2 and pFOXC3 RTs and which utilize the mitochondrial genetic code, are depicted in SEQ ID NO:5 and SEQ ID NO:6, respectively. In view of the fact that the pFOXC2 RT (SEQ ID NO:1) and the pFOXC3 RT (SEQ ID NO:2) sequences are 88% identical along their full length, preferred RTs comprise sequences that are at least 88% identical to either SEQ ID NO:1 or SEQ ID NO:2 (the “pFOXC-RTs”). FIG. 2 depicts an alignment of SEQ ID NO:1 and SEQ ID NO:2, wherein 468 out of 527 amino acids are identical along the entire length of both polypeptides.

Sequence identity or percent identity is intended to mean the percentage of amino acid residues that are identical between two polypeptide sequences that are aligned according to their primary structure. The reference sequence may be either pFOXC2-RT (depicted in SEQ ID NO:1) or pFOXC3-RT (depicted in SEQ ID NO:2). To determine percent identity, the two sequences being compared are aligned using the Clustal method of multiple sequence alignment (Higgins et al, Cabios 8:189-191, 1992), which is freely available on the NIH website or under license from commercial vendors, such as in the Lasergene biocomputing software (DNASTAR, INC, Madison, Wis.). According to this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 (Dayhoff et al., in Atlas of Protein Sequence and Structure, Dayhoff, Ed., NBRF, Washington, Vol. 5, suppl. 3, p. 345, 1978).

Percent conservation is calculated from the above alignment by adding the percentage of identical amino acid residues to the percentage of amino acid positions at which the two residues represent a conservative substitution (defined as having a log odds value of greater than or equal to 0.3 in the PAM250 residue weight table). Conservation is referenced to either pFOXC2-RT (depicted in SEQ ID NO:1) or pFOXC3-RT (depicted in SEQ ID NO:2). Conservative amino acid changes satisfying this requirement are: R-K; E-D, Y—F, L-M; V—I, Q-H.

Having discovered a novel feature of the pFOXC2 and pFOXC3 RTs, the novel feature being the ability of these RTs to utilize a non-base-paired (i.e., “mismatched”) 3′-OH nucleotide to prime DNA synthesis, it is envisioned by the inventor that these RTs have novel uses and improved activity for some applications compared to conventional RTs, such as MMLV-RT and e-AMV. For example, RNAs or DNAs can be copied such that mismatched nucleotides may be incorporated into the newly synthesized strand. It is therefore envisioned that the instant pFOXC-RTs may be useful in the execution of in vitro mutagenesis protocols, wherein a pFOXC-RT is mixed with RNAs or DNAs and random or specific polynucleotide primers. Accordingly, any mismatched nucleotides of the polynucleotide primer will be incorporated into the resultant synthetic strand.

Further, given that the genomes of many species comprise many millions of single-nucleotide polymorphisms (“SNPs”), it is also envisioned that the instant pFOXC-RTs may be useful in genomic profiling. Given the large number of SNPs for any given species, it is improbable to know the exact genomic sequence or transcriptosomic sequences for any given individual, prior to determining the exact sequence of any given individual's genome. Thus, the instant pFOXC-RTs may be used in a protocol designed to profile the expressed sequences of an individual using RT technology, wherein the exact nucleotide sequence of said individual is not known (i.e., any and all RNAs, including non-poly-adenylated transcripts and very short RNAs, may be cloned or amplified using mismatched primers due to unforeseen SNPs or loosely paired snapped back primers). Currently available RTs require an exact nucleotide complementary match at the 3 prime end of the RT primer in order to copy a DNA or RNA, and therefore have limited utility in the genetic profiling of uncharacterized individuals comprising unknown SNPs.

It is also envisioned that the instant pFOXC-RTs may be used to generate long cDNAs or polynucleotide strands in the presence of several mismatches, without the stalling of polynucleotide synthesis. The instant pFOXC-RTs are expected to be highly processive, due to their ability to incorporate mismatched base nucleotides without stalling. The rationale for this specific utility is that a mismatched nucleotide incorporated into a growing polynucleotide strand will not interrupt strand synthesis, since the instant pFOXC-RT does not require a perfectly matched 3′ nucleotide to catalyze phosphodiester bond formation. Nonetheless, as with oligonucleotide primers in general, the primer must anneal at least in part to the template polynucleotide strand during the initiation of polymerization. Those mismatched base nucleotides that are incorporated into the new strand may occur due to limited specific deoxynucleotides present in the reaction mixture. For example, if a specific complementary base nucleotide is not available for incorporation into the growing (new) strand, the pFOXC-RT may use a non-complementary base nucleotide to incorporate into the new strand, allowing for DNA synthesis to continue unimpeded. Again, it is envisioned that this attribute of the pFOXC-RTs is useful in mutagenesis as well as genomics profiling protocols.

It is also envisioned that the instant pFOXC-RT may be useful in improved methods of making cDNAs or cDNA libraries. Current methods of making cDNAs or libraries of cDNAs, which utilize MMLV-RT or other commercially available RTs, very frequently result in short or incomplete double stranded cDNAs. Incomplete or short cDNAs are thought to result not from low processivity of the RT during minus (new) strand synthesis, but rather by fortuitous snapping back of the 3 prime end of the first synthetic polynucleotide strand (minus strand) to form a primer needed by the RT for second strand synthesis (i.e., the “topping” reaction). Since (a) the commercially available RTs require stable base-pairing of the 3′ nucleotide of the primer to efficiently synthesize a polynucleotide strand, (b) the same RT is used in the topping reaction, and (c) the primer used in the topping reaction results from snapping back of the first synthesized strand onto itself, then (d) efficient second strand synthesis, and ultimately the length of the final double-stranded cDNA, depends upon the position in which a stable hairpin structure is formed along the first synthesized (minus) strand. The instant pFOXC-RTs do not require that the 3′ nucleotide be stably base paired in order for efficient polynucleotide synthesis to occur. Thus, the instant pFOXC-RTs may utilize a hairpin structure that occurs further upstream of the template (i.e., in the 5 prime direction of the template strand or the 3 prime direction of the first synthesized strand), enabling the formation of a much longer cDNA molecule. Longer cDNAs are likely to be more complete and therefore contribute to higher quality and more useful cDNA libraries.

It is also envisioned that the instant pFOXC-RT may be useful in the identification, isolation, copying or cloning of small RNAs. Recently, classes of small RNAs (<50 nts long, usually 20-21 nts) have been discovered. These “small RNAs” are known as and include double stranded RNA (“dsRNA”), small interfering RNA (“siRNA”), micro RNA (“miRNA” or “micRNA) and small temporal RNA (“stRNA”). These small RNAs are thought to be involved in the regulation of gene expression (gene silencing) and in the protection of a host genome against invasion by viral genomes, transposons or aberrant polynucleotides. Thus, the identification, isolation, copying or cloning of these small RNAs, in order to determine their molecular sequence, is an important research goal. The copying of these small RNAs with currently available polymerases and reverse transcriptases is expected to be inefficient and unreliable. Given the natural ability of the instant pFOXC-RT to (a) utilize a primer formed as the result of a polynucleotide strand snapping back upon itself to form a hair pin structure, and (b) to utilize loose primer specificity to initiate polymerization, it is envisioned and expected that the instant pFOXC-RT is more efficient at copying small RNAs for molecular analysis. Furthermore, if small RNAs were tailed with polyadenylase, then copied using oligo-dT primers and a conventional RT, the success of the copying and subsequent analysis relies on the efficiency of the polyadenylase or on the relative abundance of any specific small RNA entity. Thus, rare small RNAs may be missed entirely. By utilizing the instant pFOXC-RT, it is reasonable to expect that most, if not all, small RNA present in a sample would be copied into DNA and able to be subsequently cloned and amplified. To improve the efficiency of making cDNA from rare small RNAs, purified RNA may be size-selected prior to reverse transcription, second strand synthesis and subsequent analysis. Thus, the pFOXC-RTs embody an improved and practical method for identifying, isolating, and analyzing small RNAs.

Other fungal reverse transcriptases with unique activities are known in the art, including a RT encoded by a retroplasmid derived from Neurospora crassa. However, as is demonstrated in the following examples, the instant pFOXC-RT behaves much differently than the well-characterized RT encoded by the related Mauriceville retroplasmid of N. crassa. Those differences in activity include the following. (a) The pFOXC-RT uses the 3′OH of RNA templates to prime cDNA synthesis, whereas the Mauriceville-RT rarely uses RNA primers and appears to depend on a specific RNA sequence, rather than base-pairing of the 3′ end of the RNA primer (Wang and Lambowitz, 1993). (b) The pFOXC-RT is able to use DNA primers that anneal at an internal region of the transcript, whereas the Mauriceville-RT cannot (Wang et al., 1992; Chen and Lambowitz, 1997). (c) The pFOXC-RT is able to copy DNA templates, while the Mauriceville-RT can not. (d) Treatment of pFOXC-containing mt RNPs (mitochondrial ribonucleoprotein particles) with micrococcal nuclease results in RT preparations free of endogenous RNA or DNA, which at least are not used as primers for cDNA synthesis, whereas MN-treated Mauriceville-RT preparations contain endogenous cDNA products that are used as primers for reverse transcription (Wang et al., 1992). (e) The pFOXC-RT does not have strong specificity for RNAs, whereas the Mauriceville RT highly prefers RNAs having a 3′ terminal CCA sequence (Chen and Lambowitz, 1997).

The pFOX RT polypeptides of the instant invention may be produced in any biological expression system. Those biological systems include naturally occurring Fusarium oxysporum mitochondria and heterologous protein expression systems. As described below, naturally occurring pFOX RTs may be purified from Fusarium oxysporum cells. The purification steps comprise extracting mitochondrial ribonucleoprotein (“mt RNP”) particles from Fusarium cells, then subjecting the extracts to DEAE-Sephacyl chromatography to remove contaminating nucleases (see Walther and Kennell, 1999). In a preferred embodiment, the partially purified mitochondrial RNPs, which comprise pFOXC-RT, are further treated with a nuclease (e.g., RNase A, micrococcal nuclease) to degrade endogenous template RNAs and to release the pFOXC-RT. The polynucleotides that encode naturally occurring pFOX RT, i.e., that which is produced in the mitochondria, utilize the Mold Mitochondrial genetic code, in which tryptophan is encode by both T/UGG and T/UGA. The T/UGA codon is a stop codon in the Universal genetic code.

Generally, in order to produce pFOX RT in conventional protein expression systems, the polynucleotides which encode pFOX RT will preferably utilize the Universal genetic code. Since naturally occurring pFOX RT polynucleotides use the Mitochondrial genetic code, the T/UGA tryptophan codons must be changed to T/UGG to comply with Universal genetic code rules. Methods of mutating polynucleotides are well-known in the molecular biology arts (Molecular Cloning, Sambrook et al., 1989) and kits for performing site-directed mutagenesis are commercially available (e.g., QuikChange® XL site directed mutagenesis kit [Stratagene], The Altered Sites® II in vitro Mutagenesis System [Promega], Transformerm™ Site-Directed Mutagenesis Kit [BD Biosciences Clontech]). Alternatively, a pFOXC-RT polynucleotide that utilizes a Mitochondrial genetic code may be expressed in an E. coli strain that contains a UGA suppressor gene, thereby allowing for translation through the U/TGA codon.

Heterologous protein expression systems, which are useful in the production of pFOX RT, are also well-known in the art (see Protein Expression: A Practical Approach, Higgins and Hames, eds., 1999, Oxford University Press and references therein, which are incorporated herein by reference; and “Cookbook for Eukaryotic Protein Expression: Yeast, Insect, and Plant Expression Systems,” by Christopher Smith, The Scientist, 12 [22]:20, Nov. 9, 1998). When expressing a polynucleotide to produce a polypeptide in a heterologous protein expression system, the polynucleotide may be operably linked to a particular promoter sequence that is useful for driving transcription of the polynucleotide in that particular heterologous protein expression system. Particular promoter sequences that are useful in the practice of this invention include, but are not limited to constitutive promoter, inducible promoter, CMV promoter, alcohol dehydrogenase promoter, T7 promoter, lactose-inducible promoter, heat shock promoter, temperature-inducible promoter, tetracycline-inducible promoter, and the like. As used herein, the term “promoter” means any regulatory nucleotide sequence that controls the expression of another nucleotide sequence in cis. Promoters, as used herein, include traditional promoter sequences, enhancers, upstream activating sequences, silencer elements, and the like. It is envisioned that pFOX RTs can be produced in at least one of the following protein expression systems, using standard molecular cloning procedures, readily available vectors, and polynucleotides, such as SEQ ID NO:3-6 or fragments thereof, which encode a pFOX RT. Those protein expression systems include, but are not limited to: (1) prokaryotic systems, which include host organisms such as E. coli, Lactococcus lactis, and Bacillus spp. (for a detailed description on how to express proteins in E. coli, see Sambrook et al., Molecular Cloning, 1989, Cold Spring Harbor Press, which is incorporated herein by reference); (2) yeast expression systems, which include Pichia pastoris, Pichia methanolica, and Saccharomyces cerevisiae (for a detailed description on how to express proteins in Pichia spp., see Higgins and Cregg, Pichia Protocols, 1998, Humana Press); (3) insect cell expression systems, which include baculovirus, Schneider cells and stable recombinant cell lines, such as Insect Select™ system (Invitrogen) (see for example Invitrogen publication “Express Insect™ Kit and Vector Set” version B, cat. no. 052102, 25-0440, 2003); (4) other cell or organism based systems, including mammalian cells and associated viral vectors, transgenic mice, Xenopus oocytes, milk of transgenic animals, and transgenic plants (see Kuroiwa et al., Nature Biotechnology, 20:889-894, 2002; Dove, Alan, “Uncorking the biomanufacturing bottleneck,” Nature Biotechnology, 20:777-779, 2002; Hondred et al., Plant Physiology, 119:713-723, 1999; Fischer et al., Biotechnol. Appl. Biochem., 30:113-116, 1999; which are incorporated herein by reference); and (5) in vitro expression systems, such rabbit reticulocyte lysate, wheat gerrn extract and Escherichia coli extract.

The above disclosure describes several preferred embodiments of the invention, which must not be interpreted as limiting the scope of the invention. It is envisioned that the skilled artisan in the practice of this invention will recognize other embodiments of this invention that are not overtly disclosed herein. The invention is further illustrated by the examples described below. These examples are meant to illustrate the invention and are not to be interpreted as limiting the scope of the invention.

### EXAMPLE 1

### pFOXC Retroplasmid Transposition in vivo

Identifying the 3′ terminus of a pFOXC2 or pFOXC3 plasmid RNA is important for designing appropriate DNA constructs to generate in vitro RNAs for use in an in vitro system and was expected to provide information about the potential mechanism(s) the plasmid employs to maintain 3′ telomere-like repeats. Three models were proposed to account for the generation and maintenance of the repeats. The first model predicts that the repeats could be generated by an “RNA snapback” mechanism that occurs during transcription. Other models predict that the repeats are added during minus strand cDNA synthesis (“DNA slideback model”) or post-replication via a mechanism analogous to that catalyzed by the telomerase complex.

To identify the 3′ termini of the retroplasmid transcripts, total mitochondrial RNA was isolated from a pFOXC3-containing strain, and RNAs of approximately 1.9-2.0 kb (representing the full-length retroplasmid transcripts) were electro-eluted from denaturing agarose gels. The isolated RNAs were tailed with adenosine residues using polyA polymerase and complementary DNAs (“cDNAs”) were synthesized using MMLV-RT with an oligo-dT primer. The resulting cDNAs were amplified by anchored PCR and cloned. Twenty-eight separate clones were sequenced (Table 1). All clones were found to contain one or more copy of the 5 bp sequence previously identified at the 3′ terminus of the pFOXC2 and pFOXC3 DNAs. Furthermore, as observed with the plasmid DNAs, the number of repeats varied and, on average, the length of the RNAs was slightly shorter than their DNA templates. While it is not possible to determine the precise template for the RNAs analyzed, the observation that most RNAs are shorter than their corresponding DNA templates fails to support a model in which the generation of the repeats occurs during transcription.

A similar approach was taken to analyze the 5′ termini of minus-strand cDNA replication intermediates. In this case, minus-strand cDNAs were generated from endogenous RNA templates using mitochondrial ribonucleoprotein (“mt RNP”) particles that had been partially purified by DEAE-Sephacyl chromatography to remove contaminating nucleases (see Walther and Kennell, 1999). The reaction products from two different mt RNP preparations were separated from plasmid DNAs that are commonly associated with the RNP particles by size-selection on denaturing agarose gels. The denatured plasmid DNAs, having a hairpin structure at their upstream terminus, migrate at approximately 3.8 kb on denaturing gels, whereas the nascent minus-strand cDNAs migrate at 1.9 kb (Walther and Kennell, 1999). Once isolated, the 5′ end of the minus-strand cDNAs were copied via primer extension and the products were tailed and amplified by PCR. Twenty-seven separate clones were sequenced and the results are included in Table 1. As with the plasmid DNAs and plasmid transcripts, the minus-strand cDNAs contained the 5 bp reiteration and the number of repeats varied among the clones analyzed. A comparison of the number of repeats among the three plasmid molecules revealed that the length of the cDNAs are, on average, longer than the plasmid RNAs, and equal to or greater than the length of the plasmid DNAs. The increased length of the cDNA sequences relative to the plasmid RNAs suggests that additional sequences are added during the initial steps of reverse transcription. While the evidence is indirect, these results support models in which the synthesis of repeats occurs during reverse transcription, such as the proposed “DNA slideback” model described in Walther and Kennell, 1999.

TABLE 1

Comparison of termini of pFOXC3 DNA, RNA
and minus-strand cDNA.

| | | | |
|---|---|---|---|
| Sequence of Individual Terminal Clones¹ | DNA² | RNA | cDNA³ |

| | | | | |
|---|---|---|---|---|
| ATTAGTCTAG ATCTA ATCT-3′ | SEQ ID NO:7 | | 6 | 2 |
| ATTAGTCTAG ATCTA ATCTA ATC | SEQ ID NO:8 | | | 1 |
| ATTAGTCTAG ATCTA ATCTA ATCT | SEQ ID NO:9 | | 16 | 3 |
| ATTAGTCTAG ATCTA ATCTA ATCa | SEQ ID NO:10 | | | 1 |
| ATTAGTCTAG ATCTA ATCTA ATCTA A | SEQ ID NO:11 | 1 | | |
| ATTAGTCTAG ATCTA ATCTA ATCTA AT | SEQ ID NO:12 | 9 | | 3 |
| ATTAGTCTAG ATCTA ATCTA ATCTA ATt | SEQ ID NO:13 | | 2 | |
| ATTAGTCTAG ATCTA ATCTA ATCTA ATC | SEQ ID NO:14 | 2 | | 2 |
| ATTAGTCTAG ATCTA ATCTA ATCTA ATCT | SEQ ID NO:15 | | 4 | 9 |
| ATTAGTCTAG ATCTA ATCTA ATCTA AcCT | SEQ ID NO:16 | | | 1 |
| ATTAGTCTAG ATCTA ATCTA ATCTA ATCc | SEQ ID NO:17 | | | 1 |
| ATTAGTCTAG ATCTA ATCTA ATCTA ATCTt | SEQ ID NO:18 | | | 1 |
| ATTAGTCTAG ATCTA ATCTA ATCTA ATCTc | SEQ ID NO:19 | | | 2 |
| ATTAGTCTAG ATCTA ATCTA ATCTA ATCTA | SEQ ID NO:20 | 1 | | |
| ATTAGTCTAG ATCTA ATCTA ATCTA ATCTA A | SEQ ID NO:21 | 1 | | 1 |
| ATTAGTCTAG ATCTA ATCTA ATCTA ATCTA AT | SEQ ID NO:22 | 1 | | |

¹Nucleotides in bold lowercase indicate mismatches with the plasmid DNA
²From Walther and Kennell, 1999
³For comparison with the plasmid DNA and RNA, the complement of the minus-strand cDNA products is shown.

The sequences listed in Table 1 include several that contain single base mismatches with the previously reported plasmid DNA sequence. In most cases, mismatches are detected at the extreme 3′ end [or 5′ end of the corresponding minus-strand cDNA] and may reasonably represent a non-templated nucleotide added during transcription. It is possible that the addition occurs from of a contaminating nucleotide during the tailing step of the cloning procedure; however, mismatched nucleotides were not identified among the plasmid DNA products. All but 2 of the mismatches are associated with cDNA products, suggesting they could be due to errors associated with reverse transcription. Surprisingly, when upstream sequences were examined (up to 100 nucleotides), several additional changes were seen in nascent cDNA, which occurred in a region approximately 15-25 nucleotides downstream from the 5′ end. As these changes only were seen in cDNA products and the percentage (approximately ⅔rds) of cloned cDNA products having these changes was similar in two independent mt RNP preparations used to generate the cDNAs, it appears that the changes are introduced during minus-strand cDNA synthesis and are not an artifact of the cloning procedure.

TABLE 2
Mismatched and inserted nucleotides in minus-strand cDNAs

| | |
|---|---|
| Sequence of individual minus-strand cDNA clones¹ | #clones |

| | | |
|---|---|---|
| TTACAGCAAGTCCAATTAGTCTAG ATCTA ATCTA AT-3′ | SEQ ID NO:23 | |
| pFOXC3 DNA | | |
| TTACAGCAAGTCCAATTAGTCTAG ATCTA ATCTA ATCTA | SEQ ID NO:24 | 1 |
| ATCTt | | |
| TTAgAGCAAGTCCAATTAGTCTAG ATCTA ATCT | SEQ ID NO:25 | 1 |
| TTAgAGCAAGTCCAATTAGTCTAG ATCTA ATCTA AT | SEQ ID NO:26 | |
| TTAgAGCAAGTCCAATTAGTCTAG ATCTA ATCTA ATCT | SEQ ID NO:27 | 1 |
| TTACAGCtAAGTCCAATTAGTCTAG ATCTA ATCTA ATCa | SEQ ID NO:28 | 1 |
| TTACAGCtAAGTCCAATTAGTCTAG ATCTA ATCTA ATCTA | SEQ ID NO:29 | 1 |
| ATCc | | |
| TTACAGCtAAGTCCAATTAGTCTAG ATCTA ATCTA ATCTA | SEQ ID NO:30 | 1 |
| ATCTc | | |
| TTACAGtaCAAGTCCAATTAGTCTAG ATCTA ATCTA ATCT | SEQ ID NO:31 | 1 |
| TTACAGtaCAAGTCCAATTAGTCTAG ATCTA ATCTA ATCTA | SEQ ID NO:32 | 1 |
| TTACAGtaCAAGTCCAATTAGTCTAG ATCTA ATCTA ATCTA AT | SEQ ID NO:33 | 1 |
| TTACAtgaGCAAGTCCAATTAGTCTAG ATCTA ATCTA ATCT | SEQ ID NO:34 | 1 |
| TTAgAGCAttGTCCAATTAGTCTAG ATCTA ATCTA ATCTA | SEQ ID NO:35 | 1 |
| ATCT | | |
| TTACAGCttcGTCCctATTAGTCTAG ATCTA ATCTA ATCTA ATCT | SEQ ID NO:36 | 3 |
| TTACAGCttcGTCCctATTAGTCTAG ATCTA ATCTA ATCTA AcCT | SEQ ID NO:37 | 1 |
| TTACAGCttacgtAGTCCtATTAGTCTAG ATCTA ATCTA ATCTA ATCT | SEQ ID NO:38 | 1 |
| TTACAGCAAGTCCAATTAGTCTAGagatctg ATCTA ATCTA ATCTc | SEQ ID NO:39 | 1 |

¹For comparison with the plasmid DNA and RNA, the complement of the minus-strand cDNA products is shown. Mismatched nucleotides are indicated in bold lowercase, and insertions are underlined.

### EXAMPLE 2

### pFOXC-RT Activity in vitro

To study the mechanism of reverse transcription of the pFOX-RT, an in vitro cDNA synthesis system was developed using partially-purified pFOXC-RT and in vitro RNAs that correspond to the 3′ end of the plasmid transcript. Initially, mt RNP particles from pFOXC3-containing containing strains were treated with micrococcal nuclease to digest the endogenous RNA associated with the pFOXC-RT and following the addition of EGTA to chelate the Ca⁺⁺ cofactor, these preparations (termed MN-pFOXC-RT) were used with poly-rA/oligo-dT (or poly-rC/oligo-dG) template/primer substrates to assay for RT activity by the incorporation of radiolabeled nucleotides. Significant counts were recorded and the reaction conditions were optimized for the following variables: pH, [Mg⁺⁺], [Mn⁺⁺], [salt], and temperature. At pH 8.2, 15 mM MgCl₂, no salt, and 42° C., the specificity of reverse transcription was assessed by replacing the template/primer substrates with total mt RNA isolated from pFOXC3-containing strains. The resulting P³²-labeled cDNA products were used as probes for a Southern blot containing restriction endonuclease fragments of mt DNA from the same strain. The resultant autoradiogram revealed that the cDNA products hybridized primarily to the plasmid band, demonstrating that the RT shows specificity for the retroplasmid RNA.

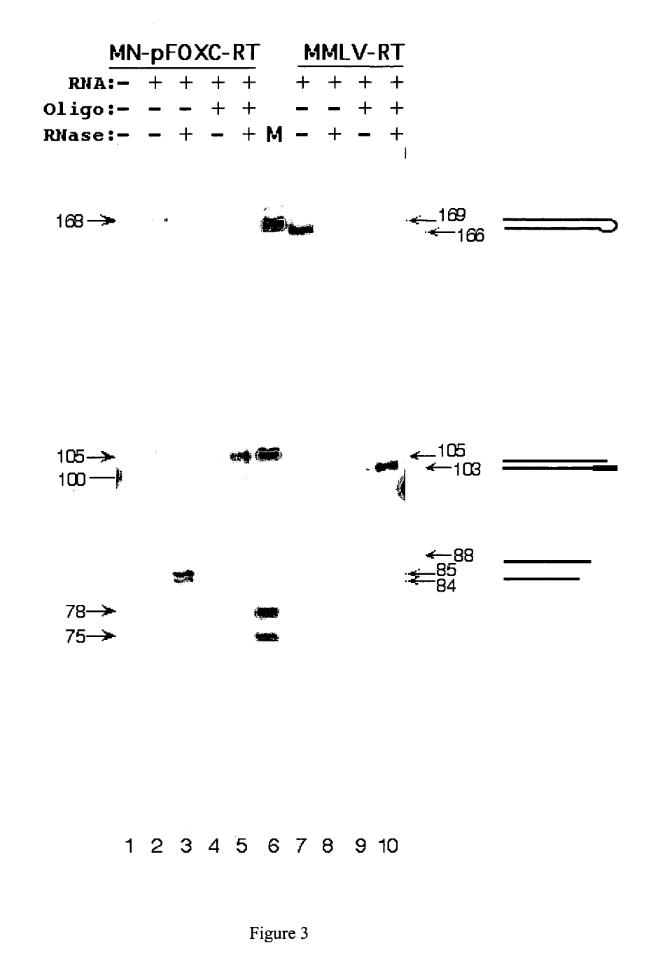

RNA templates corresponding to the 3′ end of the pFOXC3 plasmid transcript were generated by run-off transcription from DNA constructs containing the terminal ˜100 bp of the plasmid. Constructs were made so that in vitro RNAs would contain 2 or 3 copies of the 5 bp repeat, as well as other variations. Reverse transcription reactions using the MN-pFOXC-RT with in vitro RNAs were evaluated by the incorporation of radiolabeled nucleotides into cDNA products, followed by separation via electrophoresis in denaturing polyacrylamide gels. An example of a gel showing cDNA products using a 93 nt in vitro RNA having 2 copies of the 5 bp repeat is shown in FIG. 3.

In reactions containing the 93 nt RNA template (C3:2r) without added oligonucleotide primers, the cDNA products were approximately twice the length (˜169 nt) of the RNA template (FIG. 3, lane 2). When the ³²P-labeled cDNA products were digested with RNase A, bands of approximately 84-88 nts were observed (FIG. 3, lane 3). This indicated that the 169 nt band represented an RNA-DNA hybrid molecule generated by the elongation of the 3′ end of the RNA which had snapbacked upon itself. Similar, but slightly smaller products (˜166 nt) were observed when reactions were carried out using MMLV-RT. Post-treatment of the MMLV-RT cDNA products with RNase A also indicate the larger bands represent an RNA:cDNA hybrid, yet interestingly, the major cDNA products are significantly shorter (about 74 nts), compared to those obtained with the MN-pFOXC-RT. This observation indicates that the RNAs may be snapping back in alternative ways, although other mechanisms may be involved. When MMLV-RT was used in place of MN-pFOXC-RT under the same reaction conditions (higher Mg, no KCl), it did not show a snap back product, and yet it was able to extend a DNA oligonucleotide primer. In addition, the MN-pFOXC-RT was able to copy most RNAs provided in the reactions, including those that extended into the vector sequences and other RNAs that were not efficiently copied by MMLV-RT. These reactions show that the pFOXC-RT is capable of using a RNA to prime cDNA synthesis although, under the conditions tested, it appears to have little specificity for a particular RNA template.

When a 34 nt oligonucleotide (3r) having 25 nt of complementarity to the 3′ terminal sequences of the C3:2r RNA was included in the reactions, a 32P-labeled cDNA product of 105 nt was obtained, indicating that the pFOXC-RT was also capable of extending a DNA primer. Interestingly, this product is slightly larger than predicted (by 2-3 nts) and in reactions using MMLV-RT, the predicted product of 103 nt is obtained. Post-treatment with RNase A had no affect of these products.

### EXAMPLE 3

### In Vitro Biochemical Analysis of pFOXC-RT Activity Demonstrating Novel Activity

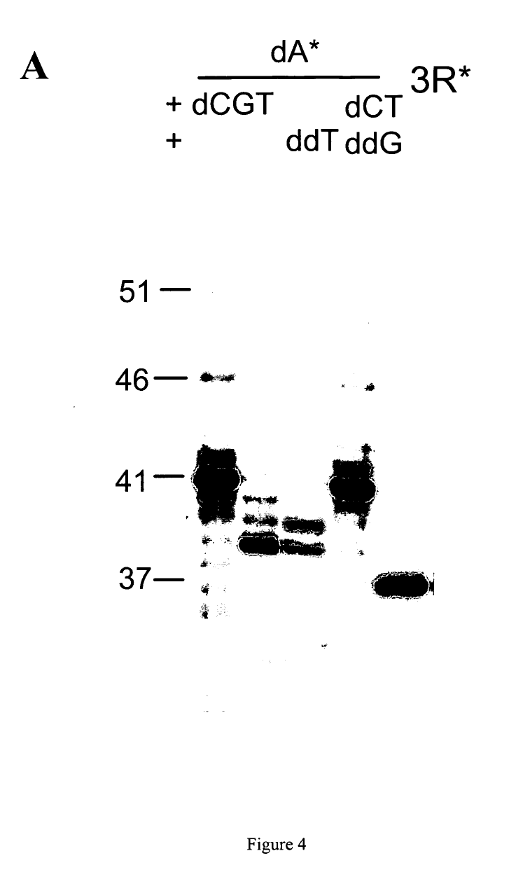

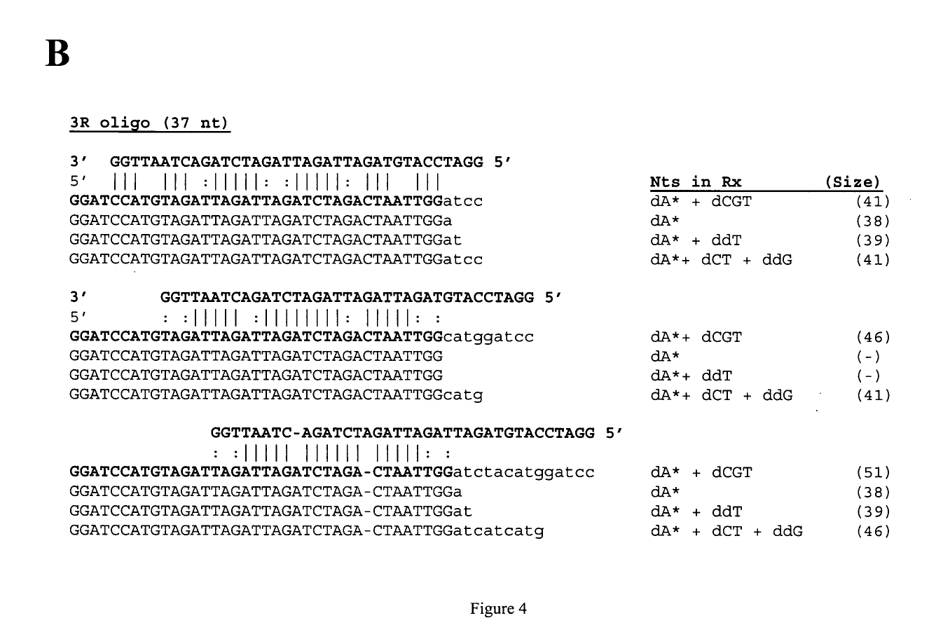

A surprising and unexpected result was found in in vitro reactions that included DNA oligonucleotides complementary to the 3′ end of the in vitro RNA templates. In addition to the cDNA products resulting from the reverse transcription reactions that used the DNA oligos as primers, labeled products were also found migrating at approximately 40-50 nts. The size of these smaller products varied slightly with the particular oligonucleotide used, yet were consistently observed and in no case were these products observed when MMLV-RT was used in equivalent reactions. Further experiments carried out without RNA templates demonstrated that the oligonucleotide used in these reactions was extended by the pFOXC-RT, using the same oligonucleotide as template. Surprisingly, the oligonucleotides were extended despite very unfavorable base-pairing interactions (ΔG of −1.6 and higher), and extension products were observed that resulted from primer/template annealing configurations in which the 3′ terminal nucleotides were mismatched. FIG. 4 shows an example of reactions carried out with a single DNA oligonucleotide of 37 nt (3R), with or without the addition of specific deoxy- or dideoxy-nucleotides that were used to characterize specific primer/template interactions. Experiments that used varying amounts of oligonucleotide primers indicate that the reactions are bimolecular, involving two primers, rather than a snapback of an individual primer. The three base pairings that appear to occur in the reaction shown in the FIG. 4 are indicated as well as the size of the predicted extension products. Subsequent experiments using end-labeled oligonucleotides having specific mismatches at the 3′ terminus confirm these interpretations.

It is noteworthy that these reactions occurred at 37° C., without the addition of potassium or sodium salts, and when MMLV-RT was used with the identical reaction conditions, no products were observed. These observations, coupled with the finding that most RNAs used in the instant in vitro cDNA system are readily copied via a snapback mechanism, indicate that the pFOXC-RT has a very relaxed specificity for primers. More significantly, the pFOXC-RT appears to be able to extend primers with mismatched 3′ nucleotides. Even considering potential G-T pairing in the oligonucleotide pairings shown in the FIG. 4, the ability to efficiently extend terminal mismatched primers is highly uncommon.

The RT associated with the Tetrahymena telomerase is the only other RT known in the art, which can efficiently extend primers having terminal 3′ mismatch (a single mismatch). However, the Tetrahymena telomerase does so only when the primers are aligned at a specific position of the RNA template (Wang et al., 1998). In the case of the pFOXC-RT, it was demonstrated that the RT can copy DNA templates, as well as RNA templates, and to extend mismatched primers.

### EXAMPLE 4

### pFOXC-RT-Specific Antibodies

Polyclonal and monoclonal antibodies are raised against epitopes of pFOXC-RT, using synthetic peptides or larger fusion proteins of the pFOXC ORF as antigen and using well-known art-recognized methods. Said antibodies are useful to determine the relative amounts of the pFOXC-RT in mt RNP particles and mitochondrial lysates. Glycerol gradient centrifugation of the pFOXC-RT preparations and subsequent Western analysis are conducted following procedures used in Kuiper et al. (1990) to determine the native size and multimeric state of the RT. 2-D gel electrophoresis separation techniques are employed to analyze the components of the mt RNP particles. In addition, an antibody is of great use in the in vitro assays to quantify the RT in reactions and to confirm the synthesis of the RT in expression studies.

### EXAMPLE 5

### Expression of pFOXC-RT in a Heterologous System

It is recognized in the art that expressing RTs in heterologous systems is very difficult and challenging, due to requirements for reverse transcription and the detrimental effects RTs can have on the host by producing highly recombinogenic (and thus mutagenic) cDNAs. The pFOXC-RT may be expressed in the yeast Ty1 retrotransposition system developed by Curcio and Garfinkel (1991), which has proven to be successful for expressing RTs encoded by several elements, including HIV (Nissley et al., 1996), hepatitis B (Qadri and Siddiqui, 1999), and human L1 (Dombroski et al., 1994). This system exploits the yeast Ty1 LTR element that retrotransposes in the yeast genome. RTs of interest are expressed as hybrids with a Ty1-encoded protein in constructs that contain the HIS3 gene in the antisense orientation. The HIS3 gene is also interrupted by an artificial intron, which is in the sense orientation of the Ty1/RT transcripts. The splicing and retrotransposition of these RNAs leads to expression of the histidine marker. Expressing clones are detected by his⁺ prototrophy on solid medium that lacks the amino acid histidine. The expression of the RT in these constructs is regulated by the Gall promoter and yeast strains are used that suppress the activity of endogenous Ty1 and Ty2 elements.

Since the RT domain of Ty1 will be replaced by the pFOXC-RT and the pFOXC plasmids are expressed using the fungal mitochondrial genetic code, extensive changes are required to ensure that RT is properly expressed in yeast. Twelve single nucleotide changes are needed to change TGA codons (normally a stop codon in the universal code) to TGG to express tryptophan. Fortuitously, several of TGA codons are clustered together and therefore only require the synthesis of ten partially overlapping 80mer oligonucleotides to construct the ORF via PCR.

The reconstructed ORF is introduced into a yeast strain having the spt3 mutation that suppresses endogenous Ty1 and Ty2. Histidine prototrophy is screened following induction with galactose. The activity of the hybrid retroelement, which comprises the pFOXC-RT activity, is measured by the frequency of retrotransposition (i.e. number of prototrophs). Immunoblots using the antibody to the pFOXC-RT are used to confirm expression. Constructs lacking critical aspartic acid residues of the RT (in the YADD catalytic core) are constructed as negative controls. This system may be exploited to assess if the pFOXC-RT is sensitive to specific RT inhibitors (i.e. ddI, 3TC) and for direct comparison to other RTs. The pFOXC-RT may be expressed in other heterologous hosts, such as bacteria, bacculovirus, mammalian cell culture or other yeasts such as Pichia spp., by more conventional methods.

### EXAMPLE 6

### Purification of Naturally Occurring pFOXC-RT from Natural Sources

Fusarium oxysporum strains used in this study were pFOXC2-containing strain 699, f. sp. raphani and pFOXC3-containing strain 725, f. sp. matthioli. These strains are maintained by H. C. Kistler (USDA-ARS Cereal Disease Lab, St. Paul, Minn.) and the Crucifer Genetics Cooperative (Department of Plant Pathology, University of Wisconsin, Madison). Strains were grown on potato-dextrose (PD) agar plates and conidia were used directly in vegetative cultures or preserved in 50% glycerol and stored at −70° C. Cornidia were germinated for 7-10 days in approximately 750 ml of 1 × Vogel's medium (Davis and de Serres, 1970) at 25° C. for isolation of mitochondria.

Mitochondria were prepared from mycelial pads by a modified flotation gradient method developed for isolation of Neurospora mitochondria (Lambowitz, 1979). Mitochondrial RNP complexes were isolated by resuspending mitochondrial pellets in 3.5 ml of HKCTD and lysed by the addition of Nonidet P-40 to a final concentration of 1%. Lysates were layered over 1.85 M sucrose cushions containing HKCTD and centrifuged in a Beckman 70.1 Ti rotor (50,000 rpm, 17 hr, 4° C.; Lambowitz, 1979 Garriga and Lambowitz, 1986). Mitochondrial RNP particles were resuspended in 1× TE [10 mM Tris-HCL (pH 7.0), 1 mM EDTA] at a concentration of 10-20 A₂₆₀ OD U/ml and stored at −70° C. To obtain nuclease-free RNP particles, mt RNPs were subjected to DEAE-Sephacel column chromatography. Approximately 2-5 A₂₆₀ OD U/ml of mt RNP particles were applied to a column containing DEAE-Sephacel (Pharmacia, Piscataway, NJ) and eluted with a step gradient of 0.25-1 M KCl. After elution with 1 M KCl, fractions were collected, combined and mt RNP particles were concentrated by centrifugation at 35,000 rpm for 4 hours at 4° C. in a Beckman 70.1 Ti rotor as described in Kennell et al. (1994).

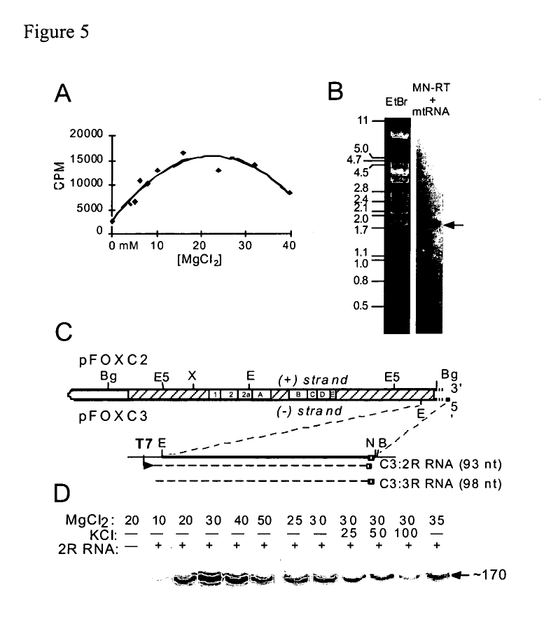

To directly study cDNA synthesis, the pFOXC-RT was liberated from mt RNP particles by nuclease treatment (supra) and reverse transcription activity was assayed using exogenous RNA-primer templates. Following steps that proved successful in the partial purification of highly-related reverse transcriptases encoded by the pMauriceville and pVarkud retroplasmids of Neurospora spp., mitochondrial RNPs from pFOXC-containing strains were treated with RNase A or micrococcal nuclease to degrade endogenous template RNAs. Reverse transcriptase activity was measured using artificial template-primers poly(rA)-oligo(dT) and/or poly(rC)-oligo(dG) with the appropriate labeled nucleotide. For most experiments, mt RNP was digested with micrococcal nuclease in the presence of 1 mM Ca++, EGTA was added to micrococcal nuclease (MN)-treated preparations to chelate the Ca⁺⁺ ions and to prevent degradation of the template-primer substrates Significant counts were detected using mt RNPs from plasmid-containing strains, and FIG. 5A shows an example of RT activity in assays carried out with different MgCl₂ concentrations using poly(rC)-oligo(dG) templates. By varying one component at a time (pH, [Mg⁺⁺], [Mn⁺⁺], [salt], and temperature), optimal reaction conditions were established to be pH 8.2, 15-20 mM MgCl₂, and incubation at 42° C. In general, the optimal conditions for endogenous RT activity using Fusarium mt RNP particles and homopolymeric template/primer substrates were very similar to previously characterized reactions using Neurospora mt RNP particles containing the Mauriceville retroplasmid (Kuiper and Lambowitz, 1988), with the slight exceptions of magnesium optimum being slightly greater (15 mM versus 10 mM) for the pFOXC-RT and requiring no salt.

The specificity of pFOXC-RT for RNA templates was assessed by replacing template/primer substrates with total mitochondrial RNA isolated from pFOXC3-containing strains. Southern hybridization experiments using the ³²P-labeled products from these reactions show that the MN-treated pFOXC-RT is specific for the plasmid transcript, as the products only anneal to a restriction band derived from the pFOXC3 plasmid (FIG. 5B). These data indicate that the purifird pFOXC-RT remains active and retains template specificity following treatment with micrococcal nuclease and demonstrate that the pFOXC-RT is amenable for use in an in vitro system that utilizes exogenous RNA substrates.

### EXAMPLE 7

### Comparison of pFOXC-RT Activity to MMLV-RT Activity

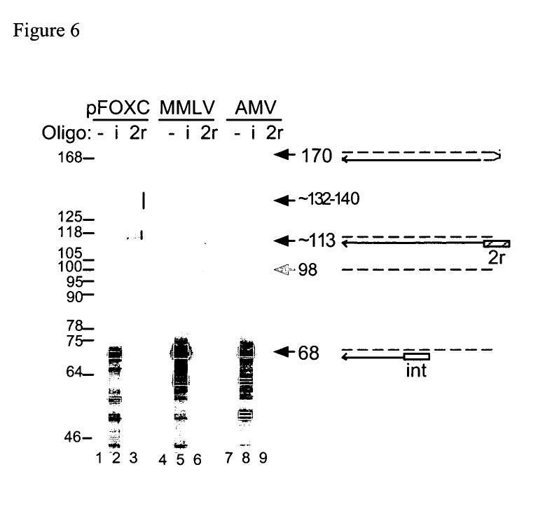

RNA templates corresponding to the 3′ end of the pFOXC3 plasmid transcript were generated by run-off transcription from DNA constructs containing the terminal ˜100 bp of the plasmid, i.e. a 93 nt RNA (C3:2r RNA) which matches the terminal 89 nt of pFOXC3 transcript plus a 5′ end containing 4 nt of the pBluescript vector. Initially, constructs were made so that in vitro RNAs would contain either 2 or 3 copies of the 5 bp repeat (C3:2R and C3:3R, respectively). Based on the supposition that the pFOXC-RT would have characteristics most similar to the Mauriceville and Varkud RTs, DNA primers were initially excluded from the reactions and cDNA synthesis using exogenous (in vitro) RNA template was monitored. Reverse transcription reactions using the MN-pFOXC-RT with in vitro RNAs were evaluated by the incorporation of radiolabeled nucleotides into cDNA products, followed by separation via electrophoresis in denaturing polyacrylamide gels. An example of a gel showing cDNA products using a 98 nt in vitro RNA having 3 copies of the 5 bp repeat is shown in FIG. 6. [Mg]

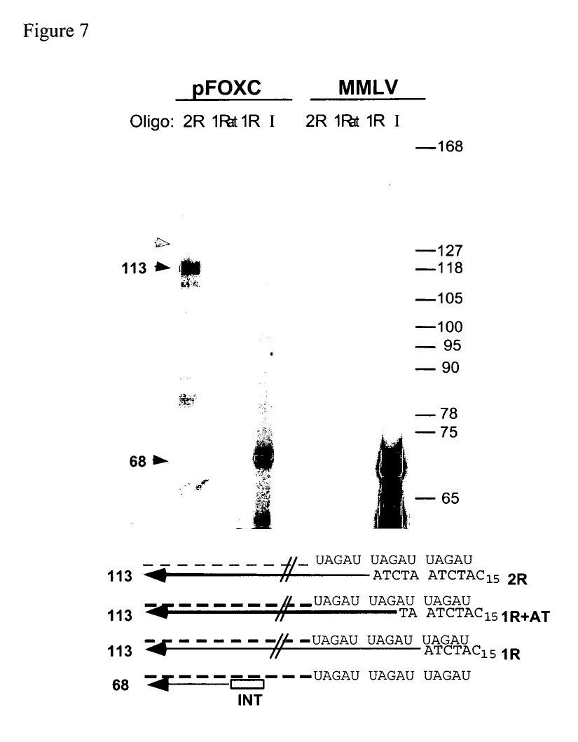

In reactions containing the 93 nt RNA template (C3:2r) without added oligonucleotide primers, the cDNA products were approximately twice the length (˜169 nt) of the RNA template (FIG. 7; lane 2). When the ³²P-labeled cDNA products were digested with RNase A, bands of approximately 84-88 nts were observed (FIG. 7; lane 3). This indicated that the 169 nt band represented an RNA-DNA hybrid molecule generated by the elongation of the 3′ end of the RNA which had snapbacked upon itself. Similar, but slightly smaller products (˜166 nt) were observed when reactions were carried out using MMLV-RT. Post-treatment of the MMLV-RT cDNA products with RNase A also indicate the larger bands represent an RNA:cDNA hybrid, yet interestingly, the major cDNA products are significantly shorter (about 74 nts), compared to those obtained with the MN-pFOXC-RT. This could suggest that the RNAs may be snapping back in an alternative ways, although other mechanisms may be involved. When MMLV-RT was used in place of MN-pFOXC-RT under the same reaction conditions (higher Mg, no KCl), it did NOT show a snap back product, and yet was able to extend a DNA oligonucleotide primer (not shown). In addition, the MN-pFOXC-RT was able to copy most RNAs provided in the reactions, including those that extended into the vector sequences and some that were not efficiently copied by MMLV-RT. These reactions show that the pFOXC-RT is capable of using a RNA to prime cDNA synthesis although, under the conditions tested, it appears to have little specificity for a particular RNA template.

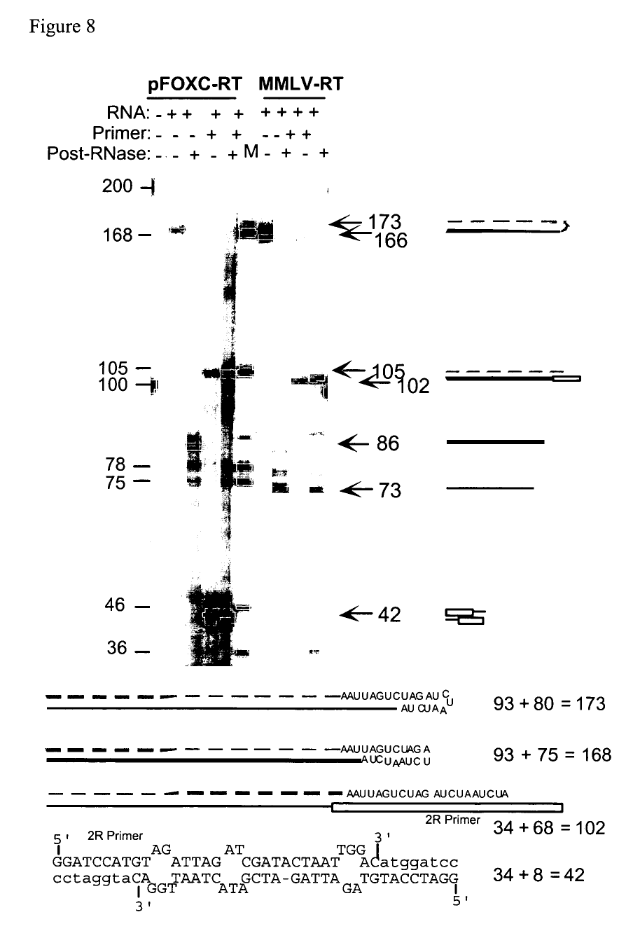

When the 93 nt C3:2r in vitro RNA was used in similar reactions (in place of the template/primers), discrete ³²P-labeled cDNA products were obtained. Resolution of these products on denaturing polyacrylamide gels revealed that they were approximately twice the length (169 nt) of the C3:2r RNA template (FIG. 8, lane 2). When the ³²P-labeled cDNA products were digested with RNase A, bands of approximately 84-88 nts were observed (lane 3). This indicated that the 169 nt band represented an RNA-DNA hybrid molecule generated by the elongation of the 3′ end of the RNA which had snap-backed on itself. A similar product was observed when identical reactions were carried out with MMLV-RT. When an 34 nt oligonucleotide having 25 nt of complementarity to the 3′ terminal sequences of the C3:2r RNA was included in the reactions, a ³²P-labeled cDNA product of 105 nt was obtained, indicating that the pFOXC-RT was capable of extending a DNA primer. Interestingly, this product is slightly larger than predicted (by 2-3 nts) and in reactions using MMLV-RT, the predicted product of 103 nt is obtained. Significantly, the ability of the pFOXC-RT to utilize a DNA primer distinguishes it from the closely related RT of the Mauriceville and Varkud retroplasmids of Neurospora.

An unexpected and quite remarkable result was found in in vitro reactions that included DNA oligonucleotides complementary to the 3′ end of the in vitro RNA templates. In addition to the cDNA products resulting from the reverse transcription reactions that used the DNA oligos as primers, labeled products were also found migrating at approximately 40-50 nts (FIG. 6). The size of these smaller products varied slightly with the particular oligonucleotide used, yet were consistently observed and in no case were these products observed when MMLV-RT was used in equivalent reactions. Further experiments carried out without RNA templates demonstrated that the oligonucleotide used in these reactions was extended by the pFOXC-RT, using the oligonucleotide as template. Surprisingly, the oligonucleotides were extended despite very unfavorable base-pairing interactions (ΔG of -1.6 and higher), and extension products were observed that resulted from primer/template annealing configurations in which the 3′ terminal nucleotides were mismatched. FIG. 6 shows an example of reactions carried out with a single DNA oligonucleotide of 37 nt (3R), with or without the addition of specific deoxy- or dideoxy-nucleotides that were used to characterize specific primer/template interactions. Experiments that used varying amounts of oligonucleotide indicate that the reactions are bimolecular, involving two primers, rather than a snapback of an individual primer. The three base pairings that appear to occur in the reaction shown in the figure are indicated as well as the size of the predicted extension products. Subsequent experiments using end-labeled oligonucleotides having specific mismatches at the 3′ terminus confirm these interpretations (data not shown).

It was further observed that pFOXC-RT uses terminal primers more readily than conventional RTs. To better assess if this is occurring in the in vitro reactions, we synthesized a DNA oligonucleotide (2c) that only has homology to the terminal 10 nts (2 copies of the 5 bp repeat) of the C3:3R RNA templates and has a homopolymeric run of C residues at the 5′ end to facilitate the recovery of the cDNA products. Equimolar amounts of RNA and primer are denatured and annealed by slow-cooling to room temperature. FIG. 8 shows a comparison of cDNA products obtained using AMV, MMLV and pFOXC reverse transcriptases at their optimal reaction conditions. [Reaction conditions for the pFOXC-RT were re-tested using the C3:2R RNA with the int primer and found to optimal at a magnesium concentration of 10 mM]. All three RTs extend an internal primer (int) that is used as a control; however, only the pFOXC-RT is able to efficiently extend the minimally base-paired 2c primer. Quantification of cDNA products indicates that the ratio of full-length cDNAs obtained from primer 2c to those of the control int reactions is more than 20 times higher with pFOXC-RT than with MMLV-RT and even greater than with AMV-RT. This is a further indication that the pFOXC-RT is highly proficient at extending loosely base-paired primers. Significantly, a higher molecular weight species is also detected in the reactions with the pFOXC-RT and the 2c primer. A group of bands that migrate 20-30 nt larger than expected are reproducibly observed in these reactions.

What is claimed is:

1. An isolated polynucleotide that comprises a sequence that encodes a reverse transcriptase polypeptide or a fragment of a reverse transcriptase polypeptide, wherein the reverse transcriptase polypeptide comprises a sequence having 88% identity to either SEQ ID NO:1 or SEQ ID NO:2.

2. The isolated polynucleotide of claim 1 wherein the polynucleotide utilizes a universal genetic code.

3. The isolated polynucleotide of claim 1 wherein the polynucleotide comprises a sequence set forth in SEQ ID NO:3 or SEQ ID NO:4.

4. The isolated polynucleotide of claim 3, wherein the polynucleotide comprises a sequence as set forth in SEQ ID NO:3.

5. The isolated polynucleotide of claim 3, wherein the polynucleotide comprises a sequence as set forth in SEQ ID NO:4.

6. The isolated polynucleotide of claim 4, wherein the polynucleotide consists essentially of a sequence as set forth in SEQ ID NO:3.

7. The isolated polynucleotide of claim 5, wherein the polynucleotide consists essentially of a sequence as set forth in SEQ ID NO:4.

8. A recombinant vector comprising a polynucleotide sequence selected from the group consisting of SEQ ID NO:3, SEQ ID NO:4, SEQ ID NO:5 and SEQ ID NO:6.

9. The recombinant vector of claim 8 wherein the polynucleotide is operably linked to a heterologous promoter.

10. The recombinant vector of claim 9 wherein the heterologous promoter is selected from the group consisting of CMV promoter, alcohol dehydrogenase promoter, T7 promoter, lactose-inducible promotes, heat shock promoter, temperature-inducible promoters, and tetracycline-inducible promoter.

11. A cell comprising an isolated polynucleotide that encodes a pFOXC-RT having a sequence that is at least 88% identical to SEQ ID NO:1 or SEQ ID NO:2.

12. The cell of claim 11 wherein the cell is selected from the group consisting of mammalian cell, mammary gland cell, plant cell, bacterial cell, yeast cell, a bacterium.

13. The cell of claim 11 wherein the cell is an Escherichia coli.

14. The cell of claim 11, wherein the cell is a Saccharomyces cerevisiae.

15. A method of making a pFOXC-RT reverse transcriptase polypeptide comprising expressing in a heterologous protein expression system an isolated polynucleotide selected from the group consisting of SEQ ID NO:3, SEQ ID NO:4, SEQ ID NO:5, and SEQ ID NO:6, wherein a pFOXC-RT is produced, and the pFOXC-RT is isolated from the heterologous system.

16. The method of claim 15 wherein the heterologous protein expression system comprises an Escherichia coli bacterial cell.

17. A method of making a complementary DNA molecule comprising (a) combining a template polynucleotide with a pFOXC-RT polypeptide, which has a sequence that is at least 88% identical to SEQ ID NO:1 or SEQ ID NO:2, in a mixture, (b) incubating the mixture in the presence of (i) MgCl₂, wherein the MgCl₂ is at a concentration in a range of 1.5 mM to 150 mM, inclusively, (ii) at a pH in a range of 6.0 to 10.0, inclusively, and (iii) at a temperature in a range of 18° C. to 54° C., inclusively, wherein (c) a new polynucleotide strand is synthesized.

18. The method of claim 17 comprising combining an oligonucleotide primer in the mixture.

19. The method of claim 18 wherein the oligonucleotide primer comprises at least one mismatched base relative to the template polynucleotide.

20. The method of claim 17, wherein the template polynucleotide is a RNA.

21. The method of claim 20 wherein the RNA is a small RNA.

22. The method of claim 17 wherein the temperature is 42° C., the MgCl₂ is at a concentration of 15 mM and the pH is 8.2.


SEQUENCE LISTING (39 sequences)

SEQ ID NO: 1 - protein, 527 aa, Fungal
MNQISKNDSLDVLQDEMGQKKTFESERKSLSGWDYFKSLGSIGRLPHFSRGIELREVKKANRYLAFQEQRIVSAIEAGEIRKAVLVWLCLMKVSRSYQILLFNRVCKGWYWRWSTARVEEIIFGAMNKMRSWDMKLLIHRFYILKKNGKMRPIGAPNYESRMISKALTDMLYSITEKSRSAEQHGYMKKRGAWSAILECLSKLKEGYAGYEFDLKSFFNTVEPFIYFRKLEEVDKKLTKLISNVIKGIEYRFSELLPESELNPKANRKNTLERTGVPQGLSLSPLLSTWALEYYGRPENLIMYADDGIYFFKHNISKFTRWIERMGRAGIEISPEKSGSLTPVFKFCGVTIDQPKRLVTYEGQSVSWDNPELEKWLKSINNLGYTKKEPEWSWTVNGESFITKRKLNLTWMEIVKVYWFRIIHGKMWNGYTVFLASGWRILDIFGSSSWACNELLMEVKRRREELESIKTFGLEKAEYEAFSYAPVRKGSYRRHYNNGAQETANKQEYWEIMQFHNLKRQQLRAIIE

SEQ ID NO: 2 - protein, 527 aa, Fungal
MNQISKNDSLDVLQDEIGQKKTFESERKSSSGWDYFKSLGSIGRLPHFNRGMELKEVKRANRYLAFQKQRIVTAVEQGEIRKAVLVWLCLMKISRSYQILLFNRVCKGWYWRWSSARVEEVIFGAMNKMRAWDMKLLIHRFYILKKNGKMRPIGAPNYESRMISKALTDLLYTVTEKSRSAEQHGYMKKRGAWSAILECLSKLKEGYAGYEFDLKSFFNTVEPFIYFRKLEEVDKKLTKVISNVIKGIEYRFTELLPESELNPKGKRKNTLVRTGVPQGLSLSPLLSTWALEYYGRPENLIMYADDGIYFFRHNISKFTRWLERMSRAGIEIAPEKSGSLKPVFKFCGVEIDQVKRLVTYDKQSVSWDNPDLEPWLKSIHNLGYTKKEPEWSWTVDQNAFITKRNLNITWMETLKVYWFRIVEGKMWNGYTVFLTSGWRVLDIFGSSSWSCNELLKIIKERKNELESIKAFGLEKAEYEAFSYAPVRKGSYRRHYNNGAPKTVNKQEYWEIMEFHNLRRQQLRAIIE

SEQ ID NO: 3 - DNA, 1584 bp, fungal
atgaatcaaatctctaaaaatgacagtttagatgttcttcaggatgaaatgggacaaaaaaagacctttgagtcagaaaggaaatctcttagtggatgggattacttcaaatcgctagggagtataggtagattaccacatttttcgagaggaattgaattacgagaagtcaagaaagcaaatagatatcttgccttccaggagcaaaggattgttagcgcaatagaagccggcgaaattcgtaaggcagtgctagtgtggctatgtttaatgaaagtctccagaagctatcagatactactattcaacagagtatgtaagggatggtactggagatggtccacggcccgggtagaagaaataatattcggggcaatgaataagatgaggtcatgggacatgaaactacttattcatagattctacatattgaagaagaatgggaagatgcgtccaataggagcaccaaactatgaatctagaatgatttccaaggcactgaccgatatgttatattcgatcactgagaaatcacgaagtgcagaacagcacgggtatatgaagaaacgtggagcctggagcgcgattctggagtgtctttcgaagttgaaagaaggatatgcaggttacgaattcgatctgaaatcgttcttcaacacggtagaaccgttcatctatttcaggaaactggaagaggtggacaagaaactgacaaagctgataagcaatgttataaaaggcatagagtacagattctcagaactactgcccgagagtgaattgaatcccaaggccaacagaaaaaatacattagagagaacgggggtgccgcaagggctgtcgctatctccactcctaagtacttgggctttagagtactatgggagaccagaaaacttaataatgtatgcagacgatggaatatacttctttaaacataatatctcaaaattcacgagatggatagagagaatgggtagagcaggtatagaaatctccccagaaaagtctggttctttgacacctgtctttaaattctgtggagtgacgattgaccaaccaaaacgattggtaacgtatgaaggacagagcgtatcatgggataacccggaactagaaaagtggctgaaaagtattaataacttggggtacacaaagaaggaaccagaatggagctggaccgtgaatggcgaatcgttcattacgaaaagaaaactgaacctcacatggatggagatagtaaaagtatactggttcagaataattcacggaaaaatgtggaatggatacactgtattccttgctagtggatggagaatccttgatatctttggatcatcatcatgggcttgtaatgagctactgatggaggtaaaaagaagaagagaagagttggaatcgattaaaaccttcggactggagaaagctgagtatgaagcattttcgtacgctccagtcagaaaaggaagttatagaaggcactataacaatggagctcaagaaacagcaaataaacaagagtattgggaaatcatgcaattccataatcttaagagacaacaactcagggctataattgagtag

SEQ ID NO: 4 - DNA, 1584 bp, fungal
atgaatcaaatctctaaaaatgacagtttagatgttcttcaggatgaaataggacaaaaaaagacctttgagtcagaaaggaaatcttctagtggatgggattacttcaaatcactggggagtatcggtagactaccacactttaacagaggtatggaattaaaggaagtgaagagagcaaatagatatcttgccttccaggagaagagaattgttactgccgtagaacagggagaaatccgtaaagctgttctcgtgtggctatgcctgatgaaaatctccagaagctatcagatcctgttattcaatagagtatgcaagggatggtactggagatggtcctctgcccgggtagaagaagtaatattcggggctatgaacaaaatgagagcatgggatatgaaactattaattcatagattctacatattaaagaagaatgggaagatgcgtcctataggagcacctaactatgaatctcgaatgatttctaaggccctaacagacctactctacactgttacggagaaatcaagaagtgcggaacagcacggatatatgaagaaaagaggagcctggagcgcgattctggaatgcctttcgaagttaaaagagggatatgcaggttatgaatttgacctaaaatcgttctttaacacagttgaaccgttcatctacttcaggaaactggaggaagtggacaaaaaactgacaaaagtgataagcaatgttataaagggcatcgagtacagattcacagagctattgcctgaaagcgagttgaatcccaagggcaaaaggaagaatacattagtgagaacgggggtgccgcaagggctgtcgttatcaccgcttctaagtacttgggctttagagtattatgggagaccagaaaacttaataatgtatgcagatgatggaatatacttctttagacataatatttcaaagttcacaagatggcttgaaagaatgtcaagagctggtatagaaatcgccccggaaaaatctggttctcttaaacctgtctttaaattctgtggagtggaaattgaccaagtcaagagactggtaacgtatgacaagcaaagtgtatcctgggataacccggatcttgaaccgtggctaaagagtatacataatttaggatacacgaagaaggaaccagaatggagctggaccgtggatcaaaatgcgttcataacgaaaagaaacttaaacatcacatggatggagactttaaaagtatactggttccgtatagttgaaggaaaaatgtggaatggatacacggtattcctgacaagtggctggagagtccttgacatctttggttcatcatcatggagctgtaatgaacttctaaagataatcaaagagaggaagaacgagttagagtctattaaggcctttggactggagaaagctgaatatgaggcgttctcatatgccccagtccggaaaggtagttatagaaggcactataacaatggagctcctaaaacagtgaataaacaggagtattgggaaatcatggaattccataacttaagaagacaacaactcagagctataattgagtag

SEQ ID NO: 5 - DNA, 1905 bp, fungal
aagctttgcttctagccattgttctaaggcttgctgtgggtcacgacactgcagctgagtagatgtccagcaaatcctcctcctcggagctttttatctagtcgaccattacgagaatcttgatttctctagggtctaaaattgttgttctaactttttatcactaacagatctaaagtgagggacaatttgaatatcagctaagaatgtaaacgaggggtatactacaccaaaccggttgttagggaagtattgctatccacaaagtaccatgaatcaaatctctaaaaatgacagtttagatgttcttcaggatgaaatgggacaaaaaaagacctttgagtcagaaaggaaatctcttagtggatgggattacttcaaatcgctagggagtataggtagattaccacatttttcgagaggaattgaattacgagaagtcaagaaagcaaatagatatcttgccttccaggagcaaaggattgttagcgcaatagaagccggcgaaattcgtaaggcagtgctagtgtgactatgtttaatgaaagtctccagaagctatcagatactactattcaacagagtatgtaagggatgatactggagatgatccacggcccgggtagaagaaataatattcggggcaatgaataagatgaggtcatgagacatgaaactacttattcatagattctacatattgaagaagaatgggaagatgcgtccaataggagcaccaaactatgaatctagaatgatttccaaggcactgaccgatatgttatattcgatcactgagaaatcacgaagtgcagaacagcacgggtatatgaagaaacgtggagcctgaagcgcgattctggagtgtctttcgaagttgaaagaaggatatgcaggttacgaattcgatctgaaatcgttcttcaacacggtagaaccgttcatctatttcaggaaactggaagaggtggacaagaaactgacaaagctgataagcaatgttataaaaggcatagagtacagattctcagaactactgcccgagagtgaattgaatcccaaggccaacagaaaaaatacattagagagaacgggggtgccgcaagggctgtcgctatctccactcctaagtacttgagctttagagtactatgggagaccagaaaacttaataatgtatgcagacgatggaatatacttctttaaacataatatctcaaaattcacgagatgaatagagagaatgggtagagcaggtatagaaatctccccagaaaagtctggttctttgacacctgtctttaaattctgtggagtgacgattgaccaaccaaaacgattggtaacgtatgaaggacagagcgtatcatgggataacccggaactagaaaagtgactgaaaagtattaataacttggggtacacaaagaaggaaccagaatggagctgaaccgtgaatggcgaatcgttcattacgaaaagaaaactgaacctcacatggatggagatagtaaaagtatactggttcagaataattcacggaaaaatgtggaatggatacactgtattccttgctagtggatgaagaatccttgatatctttggatcatcatcatgagcttgtaatgagctactgatggaggtaaaaagaagaagagaagagttggaatcgattaaaaccttcggactggagaaagctgagtatgaagcattttcgtacgctccagtcagaaaaggaagttatagaaggcactataacaatggagctcaagaaacagcaaataaacaagagtattgagaaatcatgcaattccataatcttaagagacaacaactcagggctataattgagtagactccaattacagcttagtccctattagtctagatctaatctaatctaat

SEQ ID NO: 6 - DNA, 1836 bp, fungal
gatgtccggcaaatcctcctcctcggagctttttatctaaccgacttacgggaatttcatgtccctgggtctaaaattgttcaaactttttatcactaacagatctaaagtggagacaatttgagtttcagcgaaggaaaaaaataagaggggtatactacaccaaaccggttgttagggaagttttgctatccacaaagtactatgaatcaaatctctaaaaatgacagtttagatgttcttcaggatgaaataggacaaaaaaagacctttgagtcagaaaggaaatcttctagtggatgggattacttcaaatcactggggagtatcggtagactaccacactttaacagaggtatggaattaaaggaagtgaagagagcaaatagatatcttgccttccaggagaagagaattgttactgccgtagaacagggagaaatccgtaaagctgttctcgtgtgactatgcctgatgaaaatctccagaagctatcagatcctgttattcaatagagtatgcaagggatgatactggagatgatcctctgcccgggtagaagaagtaatattcggggctatgaacaaaatgagagcatgagatatgaaactattaattcatagattctacatattaaagaagaatgggaagatgcgtcctataggagcacctaactatgaatctcgaatgatttctaaggccctaacagacctactctacactgttacggagaaatcaagaagtgcggaacagcacggatatatgaagaaaagaggagcctgaagcgcgattctggaatgcctttcgaagttaaaagagggatatgcaggttatgaatttgacctaaaatcgttctttaacacagttgaaccgttcatctacttcaggaaactggaggaagtggacaaaaaactgacaaaagtgataagcaatgttataaagggcatcgagtacagattcacagagctattgcctgaaagcgagttgaatcccaagggcaaaaggaagaatacattagtgagaacgggggtgccgcaagggctgtcgttatcaccgcttctaagtacttgagctttagagtattatgggagaccagaaaacttaataatgtatgcagatgatggaatatacttctttagacataatatttcaaagttcacaagatggcttgaaagaatgtcaagagctggtatagaaatcgccccggaaaaatctggttctcttaaacctgtctttaaattctgtggagtggaaattgaccaagtcaagagactggtaacgtatgacaagcaaagtgtatcctgggataacccggatcttgaaccgtgactaaagagtatacataatttaggatacacgaagaaggaaccagaatggagctgaaccgtggatcaaaatgcgttcataacgaaaagaaacttaaacatcacatgaatggagactttaaaagtatactggttccgtatagttgaaggaaaaatgtggaatggatacacggtattcctgacaagtggctgaagagtccttgacatctttggttcatcatcatgaagctgtaatgaacttctaaagataatcaaagagaggaagaacgagttagagtctattaaggcctttggactggagaaagctgaatatgaggcgttctcatatgccccagtccggaaaggtagttatagaaggcactataacaatggagctcctaaaacagtgaataaacaggagtattgagaaatcatggaattccataacttaagaagacaacaactcagagctataattgagtagactccaattacagcaagtccaattagtctagatctaatctaatctaat

SEQ ID NO: 7 - DNA, 19 bp, fungal
attagtctagatctaatct

SEQ ID NO: 8 - DNA, 23 bp, fungal
attagtctagatctaatctaatc

SEQ ID NO: 9 - DNA, 24 bp, fungal
attagtctagatctaatctaatct

SEQ ID NO: 10 - DNA, 24 bp, fungal
attagtctagatctaatctaatca

SEQ ID NO: 11 - DNA, 26 bp, fungal
attagtctagatctaatctaatctaa

SEQ ID NO: 12 - DNA, 27 bp, fungal
attagtctagatctaatctaatctaat

SEQ ID NO: 13 - DNA, 28 bp, fungal
attagtctagatctaatctaatctaatt

SEQ ID NO: 14 - DNA, 28 bp, fungal
attagtctagatctaatctaatctaatc

SEQ ID NO: 15 - DNA, 29 bp, fungal
attagtctagatctaatctaatctaatct

SEQ ID NO: 16 - DNA, 29 bp, fungal
attagtctagatctaatctaatctaacct

SEQ ID NO: 17 - DNA, 29 bp, fungal
attagtctagatctaatctaatctaatcc

SEQ ID NO: 18 - DNA, 30 bp, fungal
attagtctagatctaatctaatctaatctt

SEQ ID NO: 19 - DNA, 30 bp, fungal
attagtctagatctaatctaatctaatctc

SEQ ID NO: 20 - DNA, 30 bp, fungal
attagtctagatctaatctaatctaatcta

SEQ ID NO: 21 - DNA, 31 bp, fungal
attagtctagatctaatctaatctaatctaa

SEQ ID NO: 22 - DNA, 32 bp, fungal
attagtctagatctaatctaatctaatctaat

SEQ ID NO: 23 - DNA, 36 bp, fungal
ttacagcaagtccaattagtctagatctaatctaat

SEQ ID NO: 24 - DNA, 44 bp, fungal
ttacagcaagtccaattagtctagatctaatctaatctaatctt

SEQ ID NO: 25 - DNA, 33 bp, fungal
ttagagcaagtccaattagtctagatctaatct

SEQ ID NO: 26 - DNA, 36 bp, fungal
ttagagcaagtccaattagtctagatctaatctaat

SEQ ID NO: 27 - DNA, 38 bp, fungal
ttagagcaagtccaattagtctagatctaatctaatct

SEQ ID NO: 28 - DNA, 39 bp, fungal
ttacagctaagtccaattagtctagatctaatctaatca

SEQ ID NO: 29 - DNA, 44 bp, fungal
ttacagctaagtccaattagtctagatctaatctaatctaatcc

SEQ ID NO: 30 - DNA, 45 bp, fungal
ttacagctaagtccaattagtctagatctaatctaatctaatctc

SEQ ID NO: 31 - DNA, 40 bp, fungal
ttacagtacaagtccaattagtctagatctaatctaatct

SEQ ID NO: 32 - DNA, 41 bp, fungal
ttacagtacaagtccaattagtctagatctaatctaatcta

SEQ ID NO: 33 - DNA, 43 bp, fungal
ttacagtacaagtccaattagtctagatctaatctaatctaat

SEQ ID NO: 34 - DNA, 41 bp, fungal
ttacatgagcaagtccaattagtctagatctaatctaatct

SEQ ID NO: 35 - DNA, 44 bp, fungal
ttagagcattgtccaattagtctagatctaatctaatctaatct

SEQ ID NO: 36 - DNA, 45 bp, fungal
ttacagcttcgtccctattagtctagatctaatctaatctaatct

SEQ ID NO: 37 - DNA, 45 bp, fungal
ttacagcttcgtccctattagtctagatctaatctaatctaacct

SEQ ID NO: 38 - DNA, 48 bp, fungal
ttacagcttacgtagtcctattagtctagatctaatctaatctaatct

SEQ ID NO: 39 - DNA, 46 bp, fungal
ttacagcaagtccaattagtctagagatctgatctaatctaatctc